(19) United States
(12) Patent Application Publication
(10) Pub. No.: US 2013/0210896 A1
(43) Pub. Date: Aug. 15, 2013

(54) Immunotheraphy of Brain Tumors Using a Nanoparticle CpG Delivery System

(71) Applicant: CITY OF HOPE, Duarte, CA (US)

(72) Inventors: Behnam BADIE, La Canada, CA (US); Leying ZHANG, Covina, CA (US); Darya ALIZADEH, Mission Viejo, CA (US); Dongchang ZHAO, Duarte, CA (US); Jacob BERLIN, Duarte, CA (US)

(73) Assignee: CITY OF HOPE, Duarte, CA (US)

(21) Appl. No.: 13/673,973

(22) Filed: Nov. 9, 2012

Related U.S. Application Data
(60) Provisional application No. 61/557,843, filed on Nov. 9, 2011.

(51) Int. Cl. A61K 47/48

(52) U.S. Cl. CPC A61K 47/48015
USPC 514/44 R; 536/23.1

(57) ABSTRACT

One aspect of the invention relates to a nanoparticle conjugated CpG (NANO-CpG) composition and pharmaceutical composition thereof, and the preparation methods thereof. Another aspect of the invention relates to a method for using the NANO-CpG to improve CpG delivery into brain tumor associated inflammatory cells in a subject. Another aspect of the invention relates to a method for treating or preventing a method for treating and/or preventing a brain tumor in a subject comprising administering a therapeutically effective amount of the NANO-CpG or a pharmaceutical composition thereof to the subject. In one embodiment, a subject treated with the NANO-CpG composition disclosed herein or a pharmaceutical composition thereof developed immunity to brain tumor.

### PRIORITY CLAIM

This application claims priority to U.S. Application No. 61/557,843, filed Nov. 9, 2011, which is incorporated herein by reference.

### STATEMENT OF GOVERNMENT FUNDING

This work was supported by R21CA131765-01A2, James S. McDonnell Foundation (BB), ThinkCure Foundation (to BB and DJD), and P01-CA030206 and CA077544 (DJD). The City of Hope Flow Cytometry Core was equipped in part through funding provided by ONR N00014-02-1 0958, DOD 1435-04-03GT-73134, and NSF DBI-9970143.

### BACKGROUND

Brain is a common location for tumor metastasis. Approximately one third of patients with systemic cancer ultimately develop central nervous system (CNS) involvement. This is in part due to the blood-brain barrier (BBB) that prevents distribution of chemotherapeutic drugs into interstitial space and brain's “immune-privileged” status that blocks penetration of inflammatory cells into the CNS. Recent work, however, has demonstrated that CNS immunosurveillance does indeed take place both in healthy and inflammatory CNS conditions (1). Even in experimental gliomas, activated T cells have been shown to find their targets in the brain (2). These latter observations have stimulated the development of vaccine therapies against brain tumors. However, despite induction of systemic immunity, immunosuppressive tumor microenvironment itself may attenuate the host anti-tumor responses (3-4). This is highlighted in recent trials that have demonstrated poor CNS response to systemic immunotherapies (2, 5).

Local immunosuppressive tumor milieu, caused by low levels of MHC class I expression (5), production of immunosuppressive factors (6), and scarcity of antigen presenting cells have all been considered to account for poor immune responsiveness of brain tumors (3, 8 and 22). One strategy to overcome this local barrier is through activation of the innate immune system. Cells that comprise this system (e.g. microglia, macrophages, monocytes, natural killer (NK), and dendritic cells) express pattern-recognition receptors that collectively recognize macromolecules that are broadly expressed by micro-organisms. Among these, activation of toll-like receptors (TLRs) has been shown to enhance phagocytosis, promote secretion of T helper type 1 (Th1) cytokines, and mediate leukocyte recruitment to infected tissues (6-8). Accordingly, agonists such as CpG oligodeoxynucleotides (CpG or CpG ODNs) that bind TLR9 have been evaluated as cancer vaccine adjuvants and have shown some efficacy in inducing adaptive and antigen-specific cellular anti-tumor immune responses (9). However, early-stage clinical trials in patients with melanoma and gliomas have been less promising (6, 10-11). Furthermore, high doses of CpG may be toxic due to exacerbation of brain edema. Thus, there is a need to provide a novel CpG conjugation to improve CpG delivery into inflammatory cells associated with brain tumors (e.g. gliomas and metastatic brain tumor).

### SUMMARY OF THE INVENTION

One aspect of the invention relates to a method for improving CpG delivery into brain tumor associated inflammatory cells in a subject comprising administering a therapeutically effective amount of nanoparticle (NANO, e.g. carbon nanotube (CNT), gold, iron, silica, organic polymers, or carbon nanomaterials (e.g. fullerenes, rapheme, nanohorns, nanodiamond, etc.)) conjugated CpG (NANO-CpG) or a pharmaceutical composition thereof to the subject. In one embodiment, the brain tumor is gliomas. In another embodiment, the brain tumor is metastatic brain tumor.

Another aspect of the invention relates to a method for treating and/or preventing a brain tumor in a subject comprising administering a therapeutically effective amount of NANO-CpG or a pharmaceutical composition thereof to the subject.

In one embodiment, the brain tumor is gliomas. In another embodiment, the brain tumor is a metastatic brain tumor.

Another aspect of the invention relates to a composition comprising
a) a nanoparticle conjugated CpG (NANO-CpG) or a pharmaceutical composition thereof;
b) a nanoparticle modifying agent (NANOMA) conjugated CpG (NANOMA-CpG) or a pharmaceutical composition thereof; or
c) a combination of a) and b).

Another aspect of the invention relates to a type II NANO-CpG prepared by a method comprising modifying a NANO with a NANO modifying agent (NANOMA) conjugated CpG (NANOMA-CpG).

Another aspect of the invention relates to an aqueous NANO-CpG-II dispersion.

### BRIEF DESCRIPTION OF THE FIGURES

FIG. 1: Uptakes of free Cy5.5-labeled thiolated CpG (sCpG^{5.5}) and single-walled CNTs conjugated sCpG^{5.5} (CNT-sCpG^{5.5}) by primary bone marrow-derived monocytes (BMM). A) Cy5.5 signal in BMM GFP cells incubated with sCpG (0 hour); B) Cy5.5 signal in BMM GFP cells incubated with CNT-sCpG^{5.5} (0 hour); C) Cy5.5 signal in BMM GFP cells incubated with CNT-sCpG^{5.5} (12 hour); D) Cy5.5 signal in BMM GFP cells incubated with CNT-sCpG^{5.5} (12 hour); and E) sCpG^{5.5} signal in BMM GFP cells incubated with sCpG^{5.5} (left) and CNT-sCpG^{5.5} (right).

FIG. 2: A) Histogram demonstrating the uptake of CNT-sCpG^{5.5} by BMM; B) histogram demonstrating the uptake of CNT-sCpG^{5.5} by GL261 gliomas; C) histogram demonstrating the uptake of free sCpG^{5.5} by BMM; D) histogram demonstrating the uptake of free sCpG^{5.5} by GL261 gliomas; E) time course experiments showing uptake of sCpG^{5.5} and CNG-sCpG^{5.5} in BMM; and F) time course experiments showing uptake of sCpG^{5.5} and CNG-sCpG^{5.5} in GL261.

FIG. 3: Cytotoxicity of CNT-CpG measured by DAPI uptake. A) DAPI uptake of BMM incubated with CNT-sCpG^{5.5}, free sCpG^{5.5}, or PBS; and B) DAPI uptake of GL261 gliomas incubated with CNT-sCpG^{5.5}, free sCpG^{5.5}, or PBS.

FIG. 4: A) Expression of cytokines (IL-12 mRNA) in BMM incubated with CpG, sCpG, or CNT-sCpG; and B) expression of cytokines (IL-1β mRNA) in BMM incubated with CpG, sCpG, or CNT-sCpG.

FIG. 5: A) Expression of IL-12 mRNA in BMM incubated with sCpG, CNT-sCpG, CNT, or CNT and sCpG mixture (CNT+sCpG); B) expression of IL-12 in BMM incubated with sCpG, CNT-sCpG, CNT, or CNT and sCpG mixture (CNT+sCpG); C) expression of TNF-α mRNA in BMM incubated with sCpG, CNT-sCpG, CNT, or CNT and sCpG mixture (CNT+sCpG); and D) expression of TNF-α in BMM incubated with sCpG, CNT-sCpG, CNT, or CNT and sCpG mixture (CNT+sCpG).

FIG. 6: A) Expression of MCP-1 in BMM incubated with sCpG, CNT-sCpG, CNT, or CNT+sCpG; B) expression of IP-10 in BMM incubated with sCpG, CNT-sCpG, CNT, or CNT+sCpG; C) expression of MIP-1α in BMM incubated with sCpG, CNT-sCpG, CNT, or CNT+sCpG; and D) expression of VEGF in BMM incubated with sCpG, CNT-sCpG, CNT, or CNT+sCpG.

FIG. 7: A) in vivo CpG uptake by tumor-associated inflammatory GFP⁺ cells (mostly microglia and macrophages, MG/MP); B) location of intracellular CNT-sCpG (arrows) in GFP-expressing MG/MP (eGFP MG/MP); and C) representative fluorescent micrographs of the brains of the mice demonstrate persistence of sCpG^{5.5+} MG/MP at the injection sites (arrows) in animals treated with CNT-sCpG^{5.5}.

FIG. 8: A) Cy5.5 uptake in GFP⁺ tumor cells treated with CNT-sCpG^{5.5} or sCpG^{5.5}; and B) location of CNT-sCpG^{5.5} (arrows, red) in non-GFP-expressing cells (green).

FIG. 9: A) Dot plots of glioma-associated inflammatory cells (MG/MP) in GL261 tumors in w.t. mice injected with sCpG^{5.5}, B) dot plots of glioma-associated inflammatory cells (MG/MP) in GL261 tumors in w.t. mice injected with sCpG^{5.5}; C) dot plots of glioma-associated inflammatory cells (MG/MP) in GL261 tumors in w.t. mice injected with CNT+sCpG^{5.5}; D) dot plots of glioma-associated inflammatory cells (NK) in GL261 tumors in w.t. mice injected with free sCpG^{5.5}; E) dot plots of glioma-associated inflammatory cells (NK) in GL261 tumors in w.t. mice injected with CNT-sCpG^{5.5}; F) dot plots of glioma-associated inflammatory cells (NK) in GL261 tumors in w.t. mice injected with CNT+sCpG^{5.5}; G) dot plots of glioma-associated inflammatory cells (dentritic cells (DC)) in GL261 tumors in w.t. mice injected with free sCpG^{5.5}; H) dot plots of glioma-associated inflammatory cells (DC) in GL261 tumors in w.t. mice injected with CNT-sCpG^{5.5}; and I) dot plots of glioma-associated inflammatory cells (DC) in GL261 tumors in w.t. mice injected with CNT+sCpG^{5.5}.

FIG. 10: A) The proportion of sCpG^{5.5}-positive MG/MP in GL261 tumors in w.t. mice after injection with CNT-sCpG^{5.5}, free sCpG^{5.5} or CNT+sCpG^{5.5}; B) the proportion of sCpG^{5.5}-positive NK in GL261 tumors in w.t. mice after injection with CNT-sCpG^{5.5}, free sCpG^{5.5} or CNT+sCpG^{5.5}; C) the proportion of sCpG^{5.5}-positive DC in GL261 tumors in w.t. mice after injection with CNT-sCpG^{5.5}, free sCpG^{5.5} or CNT+sCpG^{5.5}; D) total inflammatory cells in GL261 tumors in w.t. mice after injection with CNT-sCpG^{5.5}; and E) CNT-sCpG^{5.5}-positive inflammatory cells in GL261 tumors in w.t. mice after injection with CNT-sCpG.

FIG. 11: A) Biophotonic imaging of mice bearing i.c. GL261.luc gliomas treated with a single intratumoral (i.t.) injection of PBS (control) to the tumor at 3, 7, and 21 days after; B) biophotonic imaging of mice bearing i.c. GL261.luc gliomas treated with a single intratumoral (i.t.) injection of free sCpG to the tumor at 3, 7, and 21 days after; C) biophotonic imaging of mice bearing i.c. GL261.luc gliomas treated with a single intratumoral (i.t.) injection of CNT+sCpG to the tumor at 3, 7, and 21 days after; and D) biophotonic imaging of mice bearing i.c. GL261.luc gliomas treated with a single intratumoral (i.t.) injection of CNT-sCpG to the tumor at 3, 7, and 21 days after.

FIG. 12: A) Kaplan-Meier analysis of survival rate of mice bearing i.c. GL261.luc gliomas treated with a single injection of PBS (control), free sCpG, CNT+sCpG, or CNT-sCpG; and B) the tumor growth rate of mice bearing i.c. GL261.luc gliomas treated with a single injection of PBS (control), free sCpG, CNT+sCpG, or CNT-sCpG.

FIG. 13: A) Kaplan-Meier analysis of survival rate of mice bearing i.c. GL261.luc gliomas treated with a single injection of PBS (control), free sCpG, CNT-sCpG or CNT conjugated with control thiolated oligodeoxynucleotide (CNT-sODN); and B) Kaplan-Meier analysis of survival rate of mice bearing i.c. GL261.luc gliomas treated with a single injection of PBS (control), free sCpG, CNT-sCpG or CNT.

FIG. 14: Effects of CNT-sCpG on tumor-associated inflammatory response in tumors treated with PBS (control), sCpG, CNT-sCpG or CpG-sODN. A) MG/MP (CD11b⁺) in brain; B) CD11b⁺/CD45⁺in blood; C) NK in brain; D) NK in blood; E) CD8⁺ in brain; and F) CD8⁺ in blood.

FIG. 15: A) Biophotonic images of intracranial tumor burden in control IgG-treated mice after tumor implantation and CNT-sCpG treatment; B) biophotonic images of intracranial tumor burden in CD8-depleted mice after tumor implantation and CNT-sCpG treatment; C) biophotonic images of intracranial tumor burden in NK-depleted mice after tumor implantation and CNT-sCpG treatment; and D) survival rates of NK-depleted mice, CD8-depleted and control IgG-treated mice after tumor implantation and CNT-sCpG treatment.

FIG. 16: A) Biophotonic images of intracranial tumor burden in CNT-sCpG-treated GL261-bearing mice and CNT-sCPG-treated GL261-bearing mice that had survived for at least three months after the initial tumor inoculation after an i.c. injection of GL261 glioma; B) biophotonic images of intracranial tumor burden in normal naïve mice after an i.c. injection of GL261 glioma; and C) survival rates of the mice.

FIG. 17: Biophotonic images of normal naïve mice (mouse 1 and 2) and CNT-sCpG-treated GL261-bearing mice that had survived for at least three months after the initial tumor inoculation (mouse 3 and 4) after a subcutaneous (s.c.) injection of GL261 glioma.

FIG. 18: In vitro NF-κB assays of CNT, CpG, and CNT-CpG.

FIG. 19: Tumor growth in mice bearing both i.c. and s.c. B16-luc melanomas treated with either intratumoral i.c. blank CNT or CNT-CpG after the initial tumor implantations.

FIG. 20: A) Tumor growth evaluation in mice bearing i.c. B16-luc melanomas treated with intratumoral i.c. CNTs (▪) or CNT-CpG () after initial tumor implantations (arrows); B) tumor size in mice bearing i.c. B16-luc melanomas treated with intratumoral i.c. CNTs (▪) or CNT-CpG () after two weeks after the initial tumor implantations; C) tumor growth in mice bearing s.c. B16-luc melanomas treated with intratumoral i.c. CNTs (▪) or CNT-CpG () after initial tumor implantations (arrows); and D) tumor size in mice bearing s.c. B16-luc melanomas treated with intratumoral i.c. CNTs (▪) or CNT-CpG () after initial tumor implantations (arrows).

FIG. 21: A) Effects of free CpG and CNT-CpG on brain tumor size in mice B) effects of free CpG and CNT-CpG on s.c. tumor size in mice; and C) effects of free CpG and CNT-CpG on the survival rates of mice bearing tumors.

FIG. 22: A) Effects of i.c. CNT-CpG treatment on melanomas in mice; and B) effects of s.c. CNT-CpG treatment on melanomas in mice.

FIG. 23: Xenogen images showing effects of i.c. and s.c. CNT-CpG therapy on tumor growth of melanomas in mice.

FIG. 24: CNT-CpG and CpG clearance in i.c. and s.c. melanomas in relation to tumor growth.

FIG. 25: A) CpG clearance in i.c. and s.c. melanomas; and B) CNT-CpG clearance in i.c. and s.c. melanomas.

FIG. 26: CNT-CpG and CpG distribution in intracranial melanomas.

FIG. 27: Characterization of tumor inflammatory cells in i.c. and s.c. melanomas after CNT-CpG treatment (B16 melanoma model). A) Proportion of leukocyte in brain tumor treated with CNT-CpG (IC or SC) or PBS (control); B) dot plots of CD11b in brain tumors after treatment with CNT-CpG (IC or SC) or PBS (control); C) dot plots of NK in brain tumors after treatment with CNT-CpG (IC or SC) or PBS (control); D) proportion of leukocyte in s.c. tumor treated with CNT-CpG (IC or SC) or PBS (control); E) dot plots of CD11b in s.c. tumors after treatment with CNT-CpG (IC or SC) or PBS (control); F) dot plots of NK in s.c. tumors after treatment with CNT-CpG (IC or SC) or PBS (control); G) Myeloid-derived suppressive cell response in brain tumor; H) dot plots of CD11b in brain tumors at baseline and after treatment with CNT-CpG (IC or SC); I) Myeloid-derived suppressive cell response in s.c. tumors; J) dot plots of CD11b in s.c. tumors at baseline and after treatment with CNT-CpG (IC or SC).

FIG. 28: Characterization of tumor inflammatory cells in i.c. and s.c. melanomas after CNT-CpG treatment (B16 melanoma model), as shown by the chromium release assay.

FIG. 29: (A) Ex vivo anti-tumor response of CpG without CNT delivery when injected into i.c. melanomas and/or into s.c. tumors for brain tumor; B) Ex vivo anti-tumor response of CpG without CNT delivery when injected into s.c. tumor i.c. melanomas and/or into s.c. tumors for s.c. tumor.

FIG. 30: A) Proportion of MG (brain), MP (SC), NK (brain) and NK (SC) in leukocytes in mice bearing i.c., s.c., or both i.c. and s.c. tumors; B) TLR9 expression in mice bearing i.c., s.c., or both i.c. and s.c. tumors; C) dot plot of CD11b in mice bearing brain tumor; and D) dot plot of CD11b in mice bearing s.c. tumor.

FIG. 31: Characterization of Lipid-PEG-LC-SPDP-CpG and SWCNT/CpG. A) ¹HNMR spectra for Lipid-PEG-LC-SPDP-CpG (top), Lipid-PEG-NH2 (middle) and RSS-CpG (bottom); B) UV-vis spectra for Lipid-PEG-LC-SPDP-CpG (top), RSS-CpG (second from the top), Lipid-PEG-LC-SPDP (third from the top) and Lipid-PEG-NH2 (bottom); C) UV-vis spectra for SWCNTs/CpG (top) and SWCNTs (bottom). TEM images of SWCNTs dispersed by Lipid-PEG-LC-SPDP-CpG/Lipid-PEG-NH2 at different ratios of D) 1:0, E) 1:1, F and G) 1:2; and H) Size distribution of SWCNT-CpG conjugates measured from FIG. 31G.

FIG. 32: In vitro activity evaluations. A) Normalized NF-κB activity of RSS-CpG, Lipid-PEG-LC-SPDP-CpG and SWCNT/CpG conjugates; B) normalized NF-κB activity of SWCNT/CpG conjugates at various SWCNT:CpG weight ratios; C) dispersal of SWCNT after 1 month storage at 4° C. (left to right: SWCNT/SDS, SWCNT/CpG, SWCNT/SDS (10:1 dilution) and SWCNTs/CpG (10:1 dilution)); and D) normalized NFκB activities of SWCNTs/CpG conjugates over time after storage at 4° C.

FIG. 33: Real-time PCR analysis with human peripheral blood mononuclear cells (PBMCs). A) IL-16; B) TNFα; C) IL-12β; and D) IL-6 mRNA.

FIG. 34: (A) Confocal images of human PBMCs after 4, 24 and 48 hours incubation with Lipid-PEG-AF555 and DSPE-PEG-LC-SPDP-CpG-Cy5.5; and B) confocal images of human PBMCs after 4, 24 and 48 hours incubation with AF555-SWCNT/CpG-Cy5.5 (AF555 was false labeled as red and Cy5.5 was false labeled as blue.)

FIG. 35: A) SWCNT-CpG eradicated established gliomas; and B) SWCNT-CpG triggered systemic immunity leading to the rejection of tumor rechallenge after 90 days.

### DETAILED DESCRIPTION

One aspect of the invention relates to a method comprising administering a therapeutically effective amount of a nanoparticle (NANO) conjugated CpG (NANO-CpG) or a pharmaceutical composition thereof to a subject, wherein the NANO-CpG administration improves the CpG delivery into brain tumor associated inflammatory cells in the subject compared to an administration of the free CpG at a same dose.

Examples of NANO include, without limitation, carbon nanotubes (CNTs), gold, iron, silica, organic polymers, carbon nanomaterials (e.g. fullerenes, graphene, nanohorns, nanodiamond, and any combinations thereof), and any combinations thereof.

As used herein, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

CNTs are allotropes of carbon with a cylindrical nanostructure (9). Examples of CNTs include, without limitation, single-walled CNTs (SWCNTs), double-walled CNTs (DWCNTs), triple-walled CNTs (TWCNTs), multi-walled CNTS (MWCNTs) and combinations thereof.

CpG or CpG ODN, is a short single-stranded synthetic DNA molecule that contains a cytosine “C” followed by a guanine “G”. The “p” refers to a phosphodiester backbone of DNA

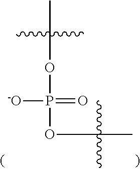

or a modified phosphorothioate backbone in a modified DNA

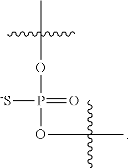

As used herein the term “conjugate” means to connect two compounds through formation of one or multiple covalent and/or non-covalent interactions. In certain embodiments, the one or multiple covalent or non-covalent interactions may be cleavable at a physiological condition of a subject. Examples of such cleavable interactions include, without limitation, cleavable covalent bonds such as bisulfide, ether, ester, amide, thio-ether, thio-ester, carbonate, carbamate, phosphate, and oxime bonds, and non-covalent interactions between DSPE and nanoparticles (e.g. CNTs).

Nanoparticles (e.g. CNTs) and CpG can be modified respectively to comprise functional groups that can form one or more covalent bonds together or can facilitate such conjugation. Such modifications can be through covalent and/or non-covalent interactions. Examples of the functional groups include, without limitation, carbonyl, carboxylic acid, acid, amine, hydroxyl, alkoxyl, and thio groups, including protonated and deprotonated forms thereof.

For example, CNTs can be modified into soluble functionalized CNTs (fCNTs) (10-12). A CpG can be modified to be a thiolated CpG (sCpG).

In certain embodiments, one or more linkers L is used to connect the NANO (e.g. CNT) and CpG. Examples of L include, without limitation, crosslinking agents that can connect the NANO (e.g. CNT) and CpG.

As used herein, a NANO-CpG conjugate may also be referred as NANO-CpG or NANO/CpG; a CNT-CpG conjugate may also be referred as CNT-CpG or CNT/CpG; and a SWCNT-CpG conjugate may also be referred as SWCNT-CpG or SWCNT/CpG.

A NANO-CpG used in the methods disclosed herein may be a type I NANO-CpG (NANO-CpG-I), a type II NANO-CpG (NANO-CpG-II), or a combination thereof.

The NANO-CpG-I is prepared by conjugating a CpG and/or a modified CpG to a modified NANO with or without L. The interactions among the CpG, modified CpG, L, and the modified NANO can be covalent and/or non-covalent interactions.

The NANO-CpG-II is prepared by providing a first CpG conjugate that can conjugate to an unmodified NANO, and then conjugating the first CpG conjugate with a NANO (modified or unmodified) to provide the NANO-CpG-II. The interactions between the first CpG conjugate and the NANO can be covalent and/or non-covalent interactions. The first CpG conjugate can be fully characterized before introduced to the NANO, thus the NANO-CpG-II is prepared with an improved characterization and reproducibility compared to the NANO-CpG-I.

In certain embodiments, the NANO-CpG-I is prepared by a method comprising the following steps:

1-a) optionally modifying a NANO to provide a NANO modified with at least a first free functional group (modified NANO);

1-b) optionally modifying a CpG to provide a CpG modified with at least a second free functional group (modified CpG); and

1-c) conjugating the modified NANO and the modified CpG to provide the NANO-CpG-I, optionally via a linker L though covalent and/or non-covalent interactions. In certain embodiments, the first and the second free function groups may be the same or different. In certain embodiments, the NANO is a CNT (e.g. a SWCNT).

In certain embodiments, the NANO-CpG-II is prepared by a method comprising the following steps:

2-a) optionally providing a NANOMA that can modify a NANO with at least a first free functional group;

2-b) optionally providing a CpG modified with at least a second free functional group (modified CpG);

2-c) optionally conjugating the NANOMA and the modified CpG to provide a NANOMA-CpG conjugate (NANOMA-CpG), optionally via a linker L though covalent and/or non-covalent interactions; and

2-d) modifying the NANO with the NANOMA-CpG to provide the NANO-CpG-II. In certain embodiments, the first and the second free function groups may be the same or different.

In certain embodiment, an aqueous NANO-CpG-II dispersion is prepared by a method comprising Steps 2-a), 2-b), 2-c) and 2-d), wherein:

Step 2-d) further comprising sonicating an aqueous mixture of NANO, NANOMA-CpG, and NANOMA.

The amount of the NANOMA needed to provide an aqueous NANO-CpG-II dispersion depends on the method used in step 2-d), and the concentrations and/or characteristics of the NANOMA, NANO and/or NANOMA-CpG (e,g. surface charges, stabilities, NANO aggregation properties). In one example, the NANO concentration is about 0.5 mg/mL or higher, about 1 mg/mL or higher, or about 2 mg/mL or higher. Optionally surfactants such as sodium dodecyl sulfate (SDS) can be used in this step to facilitate dispersal. In certain embodiments, the additional NANOMA facilitates the NANO dispersion and improve the dispersal stability, unexpectedly.

In one embodiment, the amount of additional NANOMA is at least about 1 molar eq., at least about 1.5 molar eq., or at least about 2 molar eq. of that of NANOMA-CpG. Without being bound by any specific mechanism, as it has been reported that sufficient surface charge can allow for stable dispersal of single-walled CNT (SWCNT) bundles in water (75). Thus, when CNTMA comprises one or more charged groups, these charges may be introduced to the surface of the CNT and facilitate the stable CNT dispersal.

In another embodiment, the weight ratio of NANO:NANOMA-CpG is at least about 1:10, at least about 1:5, at least about 1:2, at least about 1:1, at least about 2:1, or at least about 5:1.

In another embodiment, the CNT is a SWCNT, the CNTMA is Lipid-PEG-NH₂ as shown below:

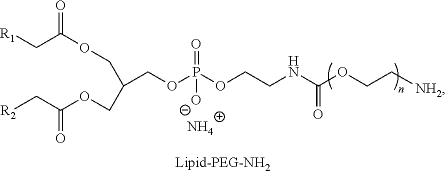

wherein:

R₁ is Cₐ alkyl;

R₂ is C_{b} alkyl;

a and b are the same or different and are independently selected from the group consisting of 10, 11, 12, 13, 14, 15, 16, 17, 18, 19, 20, 21, 22, 23, 24, 25, 26, 27, 28, 29, and 30; and

n=30, 31 . . . 44, 45, 46 . . . or 100.

A method described by Liu et al. (18) can be used to modify the SWCNT with Lipid-PEG-NH₂ to provide SWCNT-Lipid-PEG-NH₂ (Scheme 1):

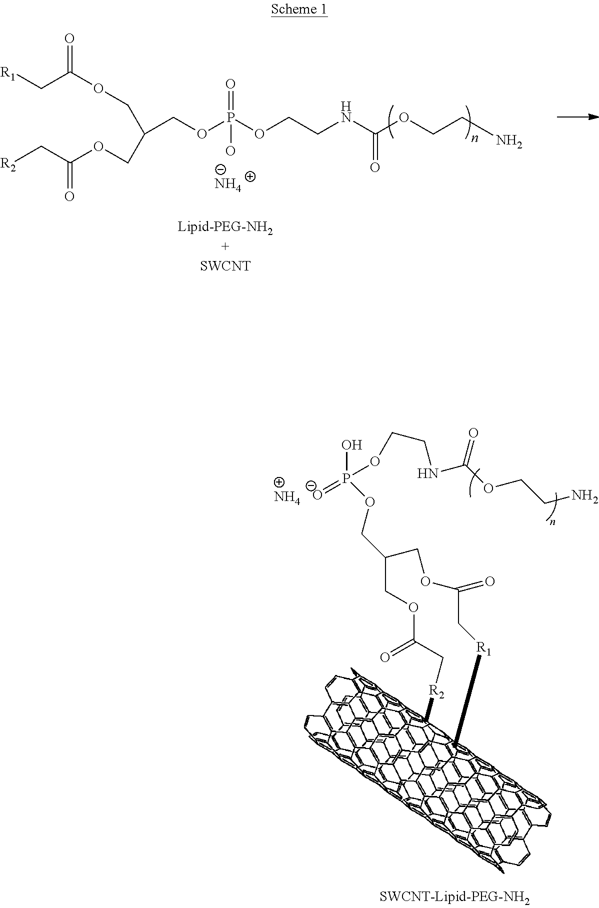

wherein the modification involves non-covalent interactions.

In another embodiment, the CpG has a sequence of SEQ ID: NO. 1 (5′-TGA CTG TAA CGT TCG AGA TGA-3′) or SEQ ID: NO. 2 (5′-TAA ACG TTA TAA CGT TAT GAO GTC AT-3′).

A CpG can be thiolated to provide a sCpG as described by Rosi et al. (12). SWCNT-Lipid-PEG-NH₂ and sCpG can be conjugated via a linker L as described by Kam et al. (19). In a preferred example, the sequence of a thiolated CpG is a thiolated sCpG having a sequence of SEQ ID: NO. 3 (5′-TGACTGTAACGTTCGAGATGA-3′) or SEQ ID: NO. 4: (5′-TAAACGTTATAACGTTATGACGTCAT-3′). The linker L is Sulfo-LC-SPDP (S-LC-SPDP) as shown below, SWCNT-Lipid-PEG-NH₂ and sCpG are conjugated through disulfide bonds to provide SWCNT-Lipid-PEG-LC-SPDP-CpG-I, which is a CNT-CpG-I (Scheme 2).

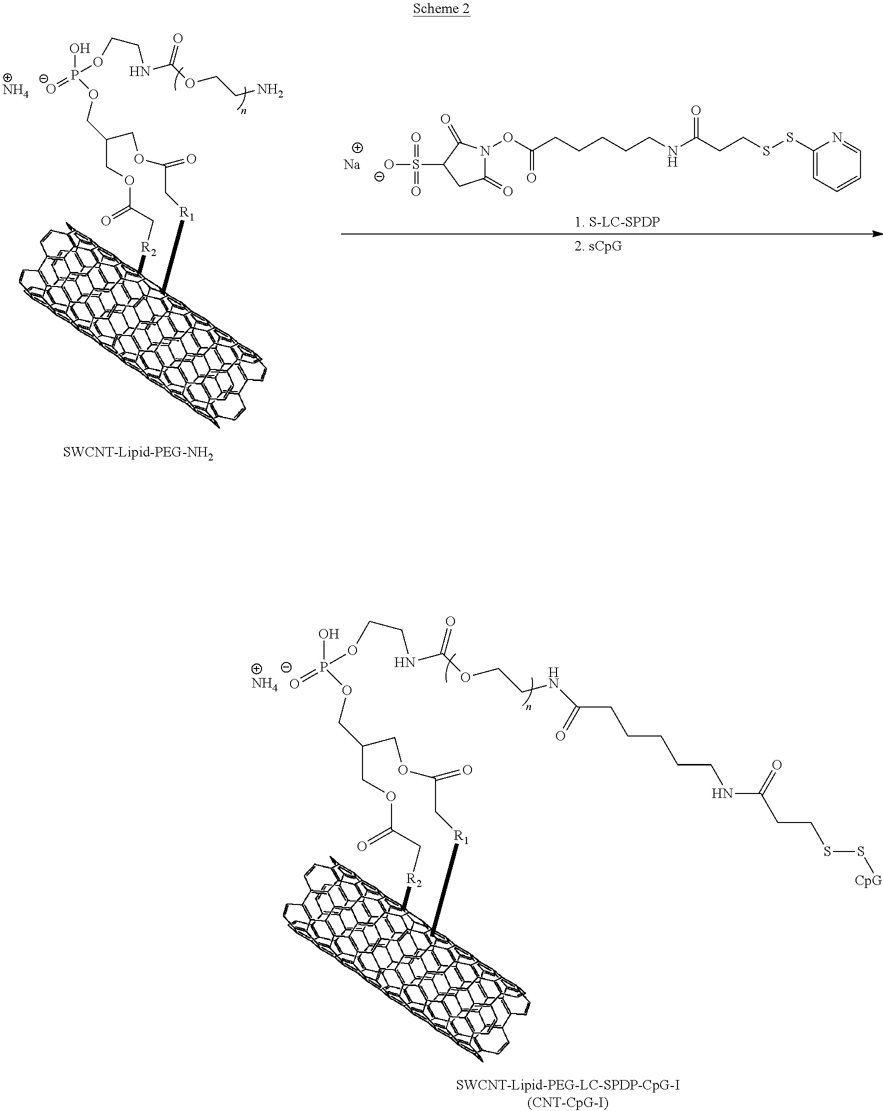

wherein the modification involves non-covalent interactions.

In one example, a=b=16, and n=45.

In another embodiment, SWCNT-Lipid-PEG-LC-SPDP-CpG-II that is a CNT-CpG-II is prepared by first reacting Lipid-PEG-NH₂ with a sCpG and a linker L (S-LC-SPDP) to provide Lipid-PEG-LC-SPDP-CpG (Scheme 3):

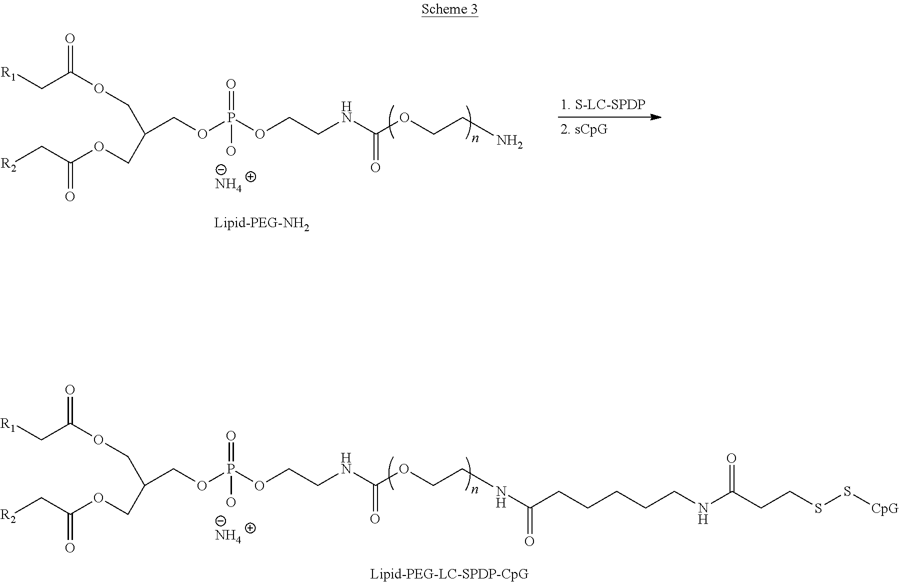

Then a SWCNT is modified with Lipid-PEG-LC-SPDP-CpG in the presence of Lipid-PEG-NH₂ (e.g. about 2 molar eq. of that of Lipid-PEG-LC-SPDP-CpG) to provide SWCNT-Lipid-PEG-LC-SPDP-CpG-II that is a CNT-CpG-II (Scheme 4):

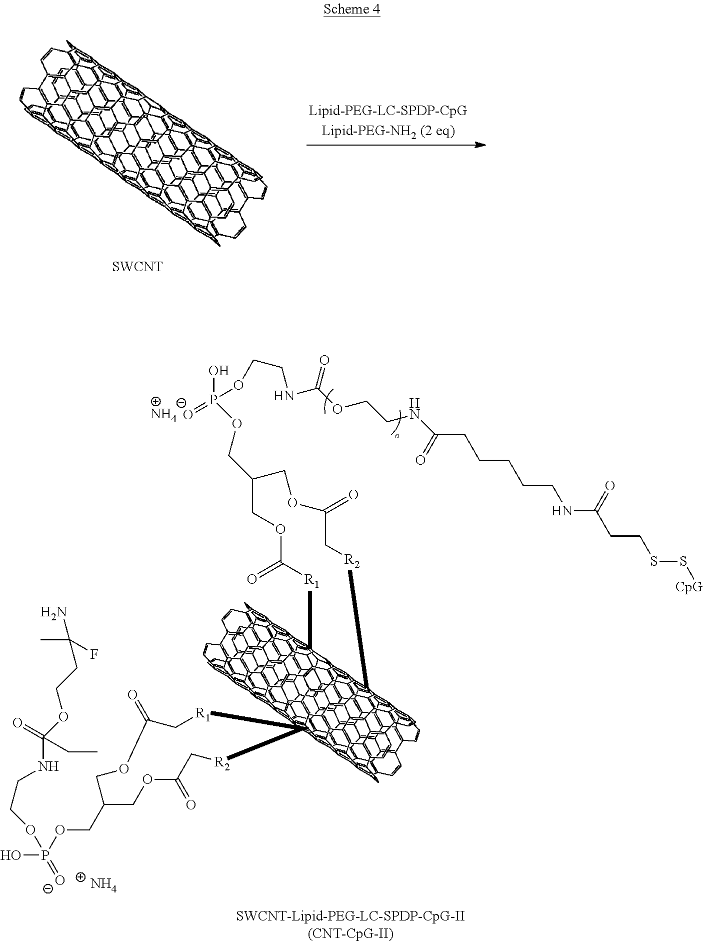

wherein the modification involves non-covalent interactions.

Examples of inflammatory cells include, without limitation, microglia and macrophages (MG/MP), NK cells, and dendritic cells (DC). Examples of inflammatory cells associated with brain tumors include, without limitation, tumor-associated MG/MP, NK cells, and DC.

A NANO-CpG can be administered to a subject by a parenteral systematic administration, and preferably an intracerebral administration.

A pharmaceutical composition of a NANO-CpG comprises the NANO-CpG and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil or injectable organic esters, or emulsions such as oil/water emulsions or a triglyceride emulsion, various types of wetting agents, tablets, coated tablets and capsules. A suitable pharmaceutically acceptable carrier may be selected taking into account the chosen mode of administration.

A pharmaceutically acceptable carrier can also contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art will know that the choice of a pharmaceutically acceptable carrier, including a physiologically acceptable compound, depends, for example, on the route of administration of the composition.

In one embodiment, the pharmaceutical carrier may be a liquid and the pharmaceutical composition would be in the form of a solution. In another equally preferred embodiment, the pharmaceutically acceptable carrier is a solid and the pharmaceutical composition is in the form of a powder or tablet.

A solid carrier can include one or more substances which may also act as flavoring agents, lubricants, solubilizers, suspending agents, fillers, glidants, compression aids, binders or table-disintegrating agents, it can also be an encapsulating material. In powders, the carrier is a finely divided solid that is in admixture with the finely divided active ingredient. In tablets, the active-ingredient is mixed with a carrier having the necessary compression properties in suitable proportions and compacted in the shape and size desired. The powders and tablets may contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Besides containing an effective amount of the NANO-CpG described herein the pharmaceutical compositions may also include suitable diluents, preservatives, solubilizers, emulsifiers, adjuvant and/or carriers.

The compound can be administered in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like.

Optimal dosages to be administered may be determined by those skilled in the art, and will vary with the particular compound in use, the severity of the disease state, drug combination(s), reaction sensitivities, and response to therapy. Additional factors depending on the particular subject being treated, including the general health of the subject, the age, weight, gender and diet of the subject, and time and frequency of administration, will result in a need to adjust dosages. Administration of the NANO-CpG or pharmaceutical composition thereof may be effected continuously or intermittently. In any treatment regimen, the NANO-CpG or pharmaceutical composition may be administered to a patient either singly or in a cocktail containing other therapeutic agents, compositions, or the like, including, but not limited to, other chemotherapies, radiation therapy, tolerance-inducing agents, potentiators and side-effect relieving agents. Preferred potentiators include temozolomide, ipilimumab (or other immune modulators), monensin, ammonium chloride, perhexyline, verapamil, amantadine, and chloroquine. All of these agents are administered in generally-accepted efficacious dose ranges such as those disclosed in the Physician's Desk Reference, 41st Ed., Publisher Edward R. Barnhart, N.J. (1987), which is incorporated herein by reference.

In the treatment, an appropriate dosage level will generally be about 0.001 to 10 mg per kg subject body weight per day that can be administered in single or multiple doses. Preferably, the dosage level will be about 0.005 to about 25 mg/kg, per day; more preferably about 0.01 to about 10 mg/kg per day; and even more preferably about 0.05 to about 1 mg/kg per day.

The frequency of dosing will depend upon the pharmacokinetic parameters of the NANO-CpG (e.g. CNT-CpG) in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data. For example, long-acting pharmaceutical compositions may be administered every 3 to 4 days, every week, or biweekly depending on the half-life and clearance rate of the particular formulation.

In one embodiment, intracerebral administration of a therapeutically effective amount of a NANO-CpG (e.g. CNT-CpG) or a pharmaceutical composition thereof showed an unexpected enhancement in the CpG uptake by tumor-associated leukocytes (See, e.g. Example 3).

Another aspect of the invention relates to a method for treating and/or preventing a brain tumor in a subject comprising administering a therapeutically effective amount of a NANO-CpG (e.g. CNT-CpG) or a pharmaceutical composition thereof to the subject. In one embodiment, the brain tumor is gliomas. In another embodiment, the brain tumor is metastatic brain tumor. Examples of the NANO-CpG include, without limitation, the NANO-CpGs described supra. Unless otherwise specified, NANO-CpG can comprise NANO-CpG-I, NANO-CpG-II or a combination thereof.

As used herein, the term “treating” means curing, alleviating, inhibiting, or preventing. The term “treat” as used herein means cure, alleviate, inhibit, or prevent. The term “treatment” as used herein means cure, alleviation, inhibition or prevention.

In one embodiment, the method disclosed herein provides an immunotherapy for brain cancer.

In another embodiment, a subject treated with the NANO-CpG composition disclosed herein or a pharmaceutical composition thereof developed immunity to brain tumor.

In another embodiment, intracerebral administration of a therapeutically effective amount of a NANO-CpG (e.g. CNT-CpG) or a pharmaceutical composition thereof unexpectedly resulted in eradication of intracranial (i.c.) gliomas in glioma-bearing mice (e.g. Example 7) and abrogated the growth of subcutaneous (s.c.) tumors in subjects bearing both i.c. and s.c. melanomas. The therapeutically effective amount of the NANO-CpG or a pharmaceutical composition thereof was unexpectedly low. The survived subjects also exhibited durable tumor-free remission and were protected from tumor rechallenge, showing induction of systemic antitumor immunity (e.g. Example 18). Thus, the intracerebral CpG therapy with NANO delivery system could be applicable to the treatment of systemic immunogenic tumors with brain metastasis.

Another aspect of the invention relates to a composition comprising
a) a nanoparticle conjugated CpG (NANO-CpG) or a pharmaceutical composition thereof;
b) a nanoparticle modifying agent (NANOMA) conjugated CpG (NANOMA-CpG) or a pharmaceutical composition thereof; or
c) a combination of a) and b).

In one embodiment, the conjugation between the NANOMA and CpG is optionally through a linker L.

In another embodiment, the composition consists essentially of:
a) a nanoparticle conjugated CpG (NANO-CpG) or a pharmaceutical composition thereof;
b) a nanoparticle modifying agent (NANOMA) conjugated CpG (NANOMA-CpG) or a pharmaceutical composition thereof; or
c) a combination of a) and b).

In another embodiment, the NANO is a CNT.

In another embodiment, the CNT is a SWCNT.

In another embodiment, the composition further comprises the NANOMA.

The amount of additional NANOMA is at least about 1 molar eq., at least about 1.5 molar eq., or at least about 2 molar eq. of that of NANOMA-CpG.

In another embodiment, the NANO-CpG is SWCNT-Lipid-PEG-LC-SPDP-CpG and the NANOMA is Lipid-PEG-NH₂ as described supra.

In another embodiment, the composition treats or prevents brain cancer.

Another aspect of the invention relates to a NANO-CpG-II as described supra, prepared by a method comprising modifying a NANO with a NANOMA-CpG.

In one embodiment, the NANO-CpG-II is a type II CNT-CpG (CNT-CpG-II).

In another embodiment, the CNT-CpG-II is a SWCNT-Lipid-PEG-LC-SPDP-CpG prepared by a method comprising the following steps:

2-a0) optionally providing a Lipid-PEG-NH₂ that can modify a SWCNT with at least a NH₂ group;

2-b0) optionally providing a sCpG;

2-c0) optionally reacting Lipid-PEG-NH₂ with the sCpG and an optional linker L (e.g. S-LC-SPDP) to provide a Lipid-PEG-L-CpG (e.g. Lipid-PEG-LC-SPDP-CpG) according to Scheme 3; and

2-d0) modifying the SWCNT with the Lipid-PEG-L-CpG in the presence of Lipid-PEG-NH₂ to provide the SWCNT-Lipid-PEG-LC-SPDP-CpG (CNT-CpG-II, Scheme 4), wherein R₁, R₂, and n are defined the same as supra.

In certain embodiments, R₁, R₂ are C₁₆ alkyl groups, and n=45.

In certain embodiments, step 2-d0) further comprising sonicating an aqueous mixture of SWCNTs, Lipid-PEG-L-CpG and Lipid-PEG-NH₂ to provide the SWCNT-CpG-II.

Another aspect of the invention relates to an aqueous NANO-CpG-II dispersion as described supra.

The amount of the NANOMA needed to provide an aqueous NANO-CpG-II dispersion depends on the method used in step 2-d), and the concentrations and/or characteristics of the NANOMA, NANO and/or NANOMA-CpG (e,g. surface charges, stabilities, NANO aggregation properties). In one example, the NANO concentration is about 0.5 mg/mL or higher, about 1 mg/mL or higher, or about 2 mg/mL or higher. Optionally surfactants such as sodium dodecyl sulfate (SDS) can be used in this step to facilitate dispersal. In certain embodiments, the additional NANOMA facilitates the NANO dispersion and improve the dispersal stability, unexpectedly.

In one embodiment, the amount of the additional NANOMA is at least about 1 molar eq., at least about 1.5 molar eq., or at least about 2 molar eq. of that of NANOMA-CpG. Without being bound by any specific mechanism, as it has been reported that sufficient surface charge can allow for stable dispersal of single-walled CNT (SWCNT) bundles in water (75). Thus, when CNTMA comprises one or more charged groups, these charges may be introduced to the surface of the CNT and facilitate the stable CNT dispersal.

In another embodiment, the weight ratio of NANO:NANOMA-CpG is at least about 1:10, at least about 1:5, at least about 1:2, at least about 1:1, at least about 2:1, or at least about 5:1.

In another embodiment, the aqueous NANO-CpG-II dispersion is an aqueous CNT-CpG-II dispersion.

In another embodiment, the aqueous CNT-CpG-II dispersion is an aqueous SWCNT-Lipid-PEG-LC-SPDP-CpG dispersion prepared by a method comprising Steps 2-a0), 2-b0), 2-c0) and 2-d0), wherein:

Step 2-d0) further comprising sonicating an aqueous mixture of Lipid-PEG-LC-SPDP-CpG, Lipid-PEG-NH₂ and the SWCNT. The suitable amount of the Lipid-PEG-NH₂ presented in Step 2-d0) should be sufficient to provide a substantially stable dispersion of the CNT-CpG-II at a desired concentration. When the concentration of the CNT-CpG-II is at least about 1 mg/mL, the amount of Lipid-PEG-NH₂ is at least about 1 molar eq. of that of Lipid-PEG-LC-SPDP-CpG, at least about 2 molar eq. of that of Lipid-PEG-LC-SPDP-CpG, at least about 3 molar eq. of that of Lipid-PEG-LC-SPDP-CpG, or from about 1 molar eq. to about 3 molar eq. of that of Lipid-PEG-LC-SPDP-CpG. When the concentration of the CNT is higher, the amount of Lipid-PEG-NH₂ needed also increases. A person of ordinary skill in the art would know how to determine the suitable amount of Lipid-PEG-NH₂ by characterizing the CNT-CpG-II prepared using different ratio among CNT, Lipid-PEG-NH₂, and Lipid-PEG-LC-SPDP-CpG. For an aqueous CNT-CpG-II dispersion having about 1 mg/mL CNT, the preferred weight ration between CNT and Lipid-PEG-LC-SPDP-CpG is at least about 1:10, at least about 1:5, at least about 1:2, at least about 1:1, at least about 2:1, at least about 5:1, or at least about 10:1.

In certain embodiments, the aqueous CNT-CpG-II dispersion as described supra is unexpectedly more stable than SDS-dispersed SWCNT at an unexpectedly high concentration (see, Example 16, wherein sonicating the SWCNT (1 mg/mL) with a 2:1 molar ratio of Lipid-PEG-NH₂ to Lipid-Peg-LC-SPDP-CpG provided an aqueous dispersion of CNT-CpG-II that was stable for over four months, with no change in activity). While a stable aqueous dispersion of the type I CNT-CpG (CNT-CpG-I)has a concentration of not more than 0.25 mg/mL. Thus, the CNT-CpG-II prepared according to the methods disclosed herein provides an unexpected more stable aqueous dispersion of CNT-CpG conjugate at an unexpected high concentration.

Furthermore, the production of the CNT-CpG-II is better characterized than that of the CNT-CpG-I, and the product of the CNT-CpG-II is better characterized than that of the CNT-CpG-I. In the preparation of the CNT-CpG-I, the final step is the conjugation of the modified CpG with the modified CNT via the linker L. The modified CNT may only react with L (CNT-L) and does not conjugate with CpG to provide the desired CNT-CpG. The amounts of CNT-L and CNT-CpG cannot be characterized. Furthermore, CNT may absorb unreacted modified CpG and/or linker L, which cannot be characterized either. Thus, it is difficult to provide a reasonable characterization of the final product. To the contrary, in the preparation of the CNT-CpG-II, the final reaction is the modification of the CNT with the CNTMA-CpG in the presence of CNTMA. The CNT is modified with one or more CNTMA-CpG and/or CNTMA, which can be better characterized. Thus, the CNT-CpG-II prepared according to the methods disclosed herein provides an unexpected better aqueous dispersion of CNT-CpG conjugate at an unexpected high concentration with a better characterization and reproducibility.

### EXAMPLES

### General Materials and Methods for Example 1-12

A thiolated CpG (SEQ ID NO:5: sCpG: 5′-TGACTGTAACGTTCGAGATGA-3′) and thiolated control oligodeoxynucleotides (sODN: 5′-TGACTGTAAGGTTAGAGATGA-3′, SEQ ID:NO. 6) were constructed as described by Rosi et al. (12) and labeled with Cy5.5 (Lumiprobe, LLC). Anti-NK1.1 (clone PK136) was purchased from eBioscience (eBioscience Inc., San Diego, Calif.). Anti-CD8 Ab (clone H35) was purified as previously described (13). Control normal mouse IgG was purchased from Santa Cruz Biotechnology (Santa Cruz Biotechnology Inc., Santa Cruz, Calif.). All flow Abs (i.e. CD11b, CD45, CD11c, CD8, and NK1.1) and isotype controls were purchased from BD Biosciences (San Jose, Calif.) or eBiosciences (San Diego, Calif.).

Cell Lines

eGFP and luciferase-expressing GL261 glioma cell lines (GL261.gfp and GL261.luc) were generated as described before (14-15). Primary bone marrow-derived monocytes (BMM) were harvested from normal C57BL/6 or CX3CR1^{GFP} mice. After washing the bone marrow with cold PBS, cells were isolated and collected with Cell Strainer (BD Biosciences, San Jose, Calif.). The isolated BMM were then cultured in L929-conditioned DMEM medium. Red blood cells and other non-adherent cells were removed by changing the culture medium in 24 hours. Cultures with more than 90% CD11b⁺ purity (as assessed by FACS) were used for experiments.

Single-Walled Carbon Nanotubes Construction and Functionalization

Single-walled carbon nanotubes (SWCNTs or CNTs) measuring 200-400 nm in length were generated and characterized by electron microscopy as described before (16-17). CNT functionalization was performed using methods described by Liu et al. (18). Briefly, hipco CNTs were sonicated extensively (1 hour) in a solution of 1,2-distearoyl-Sn-glycero-3-phosphoethanolamine-N-[amino (polyethylene glycol) 2000] (PEG) (Avanti Polar Lipids, Alabama). The supernatant solution of PEG-CNT was collected after centrifuge at 24,000 g for 6 hours. After removal of the excess PEG molecules by Amicon centrifugal filter device (100 kDa), functionalized PEG-CNTs were conjugated with Sulfo-LC-SPDP (Thermo Fisher Scientific Inc., USA) for 1 hour at room temperature. After removal of the excess Sulfo-LC-SPDP by Amicon centrifugal filter device (100 kDa) (Millipore, Billerica, Mass.), the CNT conjugates were quantified by SpectraMax M2 (Sunnyvale, Calif., USA) spectrometer with a weight extinction coefficient of 0.0465 l mg⁻¹ cm⁻¹ at 808 nm. CNTs were then conjugated with sCpGs or sODNs through a cleavable disulfide bond at 4° C. for 24 hours. Free sCpGs were then separated from the solution by Amicon centrifugal filter device (100 kDa) (Millipore, Billerica, Mass.) and measured with NanoDrop 1000 Spectrophotometer (Thermo Scientific). The CNT-bound sCpGs were quantified by subtracting the unbound sCpG from the total sCpG added prior to the conjugation reaction.

Before the CNTs were used as drug carriers, they were functionalized using a modified noncovalent technique reported by Kam et al. and linked to the CpG (19).

Real-Time PCR (RT-PCR)

BMM (5×10⁴ cells/well in 24-well plates) were incubated with sCpG (5 pg/well), CNT-sCpG (CNT 2.5 μg-sCpG 5 μg/well), blank CNT (2.5 μg/well), or CNT+sCPG mixture. At various times, cells were collected and the total RNA was generated using the Trizol system (Invitrogen Carlsbad, Calif.) followed by double DNase treatment and column purification using the Qiagen RNeasy Clean-up Protocol. Real-time PCR was performed in a TaqMan 5700 Sequence Detection System (Applied Biosystems, Foster City, Calif.) as described previously (25). The PCR conditions were optimized such that a minimum of 10,000 fold range could be detected for each primer.

GAPDH:
(SEQ ID NO: 7)
5-GTTAGTGGGGTCTCGCTCTG-3,
(SEQ ID NO: 8)
5-GGCAAATTCAACGGCACA-3;
TNF-α:
(SEQ ID NO: 9)
CAGACCCTCACACTCAGATCATCT,
(SEQ ID NO: 10)
CCTCCACTTGGTGGTTTGCTA;
IL-12:
(SEQ ID NO: 11)
5-AGAGGTGGACTGGACTCCCG-3,
(SEQ ID NO: 12)
5-AGTCTCGCCTCCTTTGTGGC-3;
and
IL-1β:
(SEQ ID NO: 13)
5-AGGGCTGTCTGGAGTCCTC-3,
(SEQ ID NO: 14)
5-GACCAGCCGCCGCCGCAGG-3.

Cytokine Multiplex Analysis

Supernatants from PCR samples were analyzed for 20 cytokines using mouse Cytokine twenty-Plex Antibody Bead Kit (Invitrogen, Camarillo, Calif.) as per the manufacturer's protocol. Assay plates were analyzed by a Bioplex HTF Luminex reader (Bio-Rad Laboratories, Inc., Hercules, Calif.) instrument. Cytokine concentrations were calculated using Bio-Plex Manager 3.1 software with a five parameter curve-fitting algorithm applied for standard curve calculations for duplicate samples. Data from detectable cytokines and chemokines were compared between each group.

In Vitro NF-κB Assay:

RAW MP cells (RAW-Blue™) stably transfected with a reporter construct expressing a secreted embryonic alkaline phosphatase (SEAP) gene under the control of a promoter inducible by the transcription factors NF-κB and AP-1 (InvivoGen) were used to measure TLR-9 activation. Upon TLR stimulation, RAW-Blue™ cells induce the activation of NF-κB and AP-1, and subsequently the secretion of SEAP. SEAP activity will be evaluated after treatments with: saline (control), S-CpG (free thiolated CpG), scrambled oligodeoxynucleotide sequence (ODN control), blank nPs, or nPs loaded with either S-CpG (nP-CpG) or scrambled sequence (nP control).

Tumor Implantation, Treatment, and Imaging

All animals were housed and handled in accordance to the guidelines of City of Hope Institutional Animal Care and Use Committee (IACUC). Intracranial tumor implantation was performed as described previously (20). GL261.luc or GL261.gfp cells were harvested by trypsinization, counted, and resuspended in PBS. Female C57BL/6 or CX3CR1^{GFP} mice that express EGFP under control of the endogenous Cx3cr1 locus (Jackson Laboratory, Bar Harbor, Me.) weighing 15-25 g were anesthetized by intraperitoneal (i.p.) administration of ketamine (132 mg/kg) and xylazine (8.8 mg/kg), and immobilized in a stereotactic head frame. Through a small burr hole, 3 μl of PBS containing 1×10⁵ tumor cells was injected unilaterally as described before (20).

Four days after i.c. tumor implantation, mice received one i.t. injection of PBS (control, 10 μL), free sCpG (5 μg/10 μL PBS), blank CNT (2.5 μg) mixed with free sCpG (CNT+sCpG; 5 μg sCpG/10 μL PBS), or sCpG conjugated to CNT (CNT-sCpG; 2.5 μg CNT/5 μg sCpG/10 μL PBS) through the initial burr hole. Tumor growth was assessed by Xenogen IVIS In Vivo Imaging System (Xenogen, Palo Alto, Calif.) as previously described (14).

NK and CD8 depletion studies were carried out as described (14). In these experiments mice were injected with anti-CD8, anti-NK1.1, or control IgG (200 pg/mouse, i.p.) mAb one day prior to tumor implantation and each i.t. injection. Leukocyte depletion was confirmed with FACS analysis of peripheral blood (14). For tumor rechallenge experiments, naive or GL261-bearing mice that had survived for at least three months after the initial CNT-sCpG treatment and were tumor-free by imaging, were re-challenged with i.c. GL261 (1×10⁵ cells).

In Vivo Uptake and Biodistribution Studies

Tumor-bearing w.t. or CX3CR1^{GFP} mice were injected with CNT bound to Cy5.5-labeled sCpG (CNT-sCpG^{5.5}, 2.5 μg CNT/5 μg sCpG/10 μL PBS) or free sCpG^{5.5} (5 μg sCpG/10 μL PBS). Tumors were harvested at various time intervals and evaluated by flow cytometry as described below. For imaging, frozen brain sections were embedded in O.C.T. (Tissue-Tek) and 10 μm sections were cut using cryostat (Leica Microsystem Inc., Bannockburn, Ill.). Sections were mounted in Vectashield mounting medium containing 4060-diamidino-2-phenylindole (DAPI) (Vector, Burlingame, Calif.). Images were obtained by AX-70 fluorescent microscopy (Leica Microsystems Inc., Bannockburn, Ill.) and were prepared by Zeiss LSM Image Browser software.

Chromium Release Cytotoxicity Assay

Cytotoxicity against B16.F10 melanoma cells was determined using a standard ⁵¹Cr release assay (16). Briefly, effector cells were derived from spleens of B16.F10-bearing C57BL6 mice (n=4) treated with either i.c. or s.c. CNTCpG. CNT-CpG injections were given 3 times, 4 days after initial tumor implantation, and every subsequent 3 days. Mice were sacrificed 48 hours after the final treatment and splenocytes (effectors) were harvested and co-incubated with irradiated (30,000 rad) B16.F10 cells for 7 days. Effectors were then co-incubated for 6 hours with 5,000 ⁵¹Cr-loaded B16.F10 targets in 96-well plates at ratios of 100:1, 20:1, and 4:1 (in triplicate). Radioactivity released into the supernatant was measured using a Cobra Quantum gamma counter (PerkinElmer). Percent specific lysis was calculated as: (experimental release−spontaneous release)/(maximum release−spontaneous release)×100%.

Flow Cytometry Analysis

Tumors and blood samples were harvested and examined by flow cytometry as described previously (14). Cell suspensions from brain tissue were forced through a 40 μm filter. Blood samples were incubated in Gey's buffer (pH 7.2) for 10 min. Freshly-prepared samples were resuspended in 0.1 M PBS containing 1% FBS and 2 mM EDTA and incubated with FcγIII/IIR-specific Ab to block nonspecific binding. Samples were then stained with different combinations Abs or isotype controls for 1 h at 4° C. and analyzed by a CyAn fluorescence cell sorter (BDIS, San Jose, Calif.). Inflammatory cells were gated and separated from the rest of sorted cells base on forward vs. side-scatter analysis and their staining characteristics. FlowJo 8.4.7 software (Tree Star, Inc., Ashland, Oreg.) was used for data analysis and the proportion of each cell type was measured as percent of total inflammatory cells. Glioma MPs were gated as CD11b⁺/CD45^{high} and MG as CD11b⁺/CD45^{low} based on previously-described phenotype characterization (21).

G-MDSC have a phenotype of CD11b1Ly6G1Ly6Clow, whereas MMDSC have a phenotype of CD11b1Ly6G_Ly6Chigh [23, 24].

Statistical Analysis

Statistical comparison in all different experimental conditions was performed with the prism software using two-way analysis of variance (ANOVA) or Student's t-test. Survival was plotted using a Kaplan-Meir survival curve and statistical significance was determined by the Log-rank (Mantel-Cox) test. A P value of less than 0.05 was considered significant.

### Example 1

### CNTs Enhance CpG Uptake In Vitro

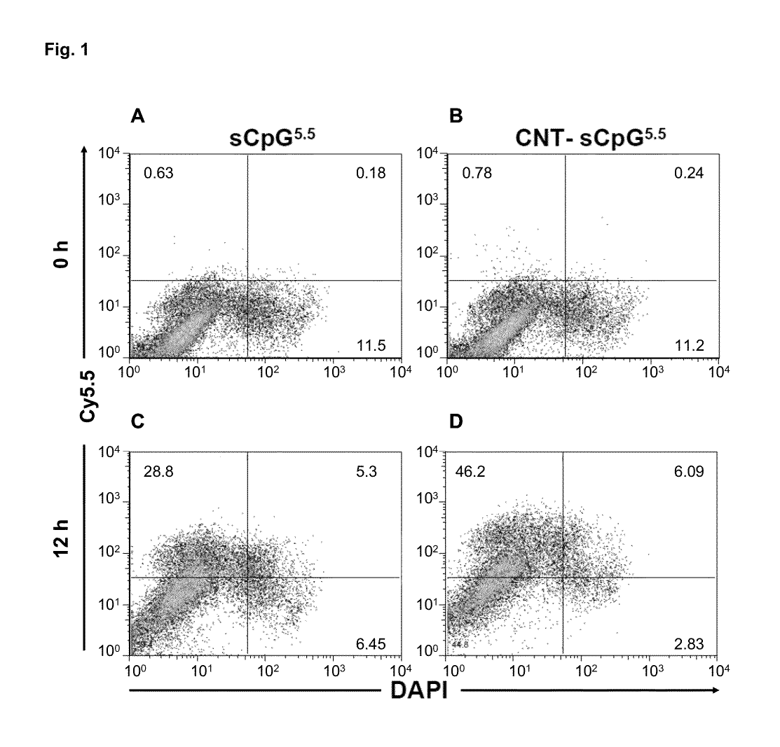

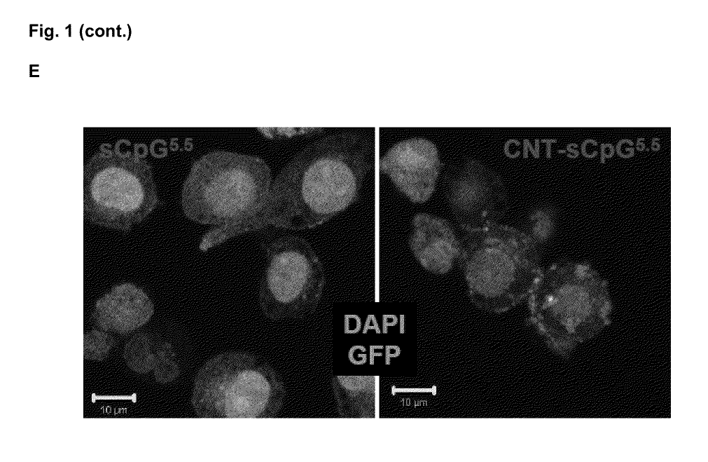

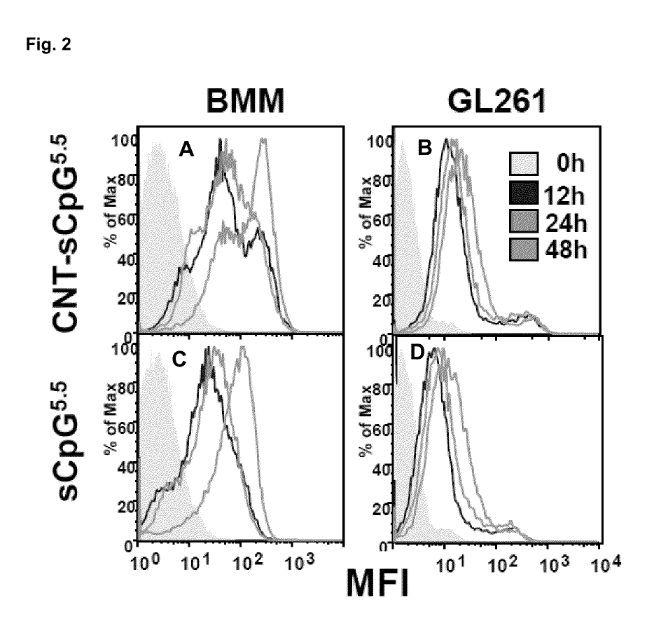

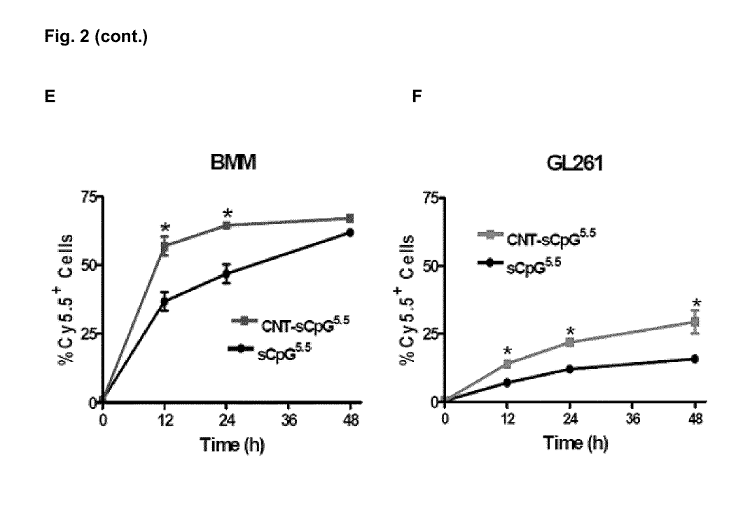

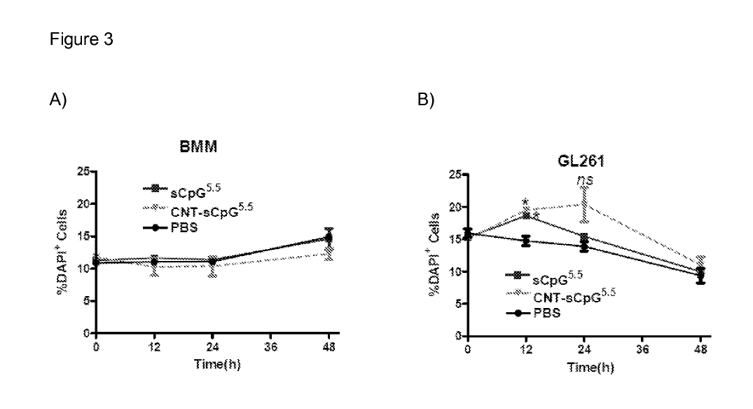

CNT conjugation capacity plateaued at 1-2 μg sCpG per pg CNT, which was consistent with other oligonucleotides (24). To evaluate sCpG uptake in vitro, BMM derived from w.t. or CX3CR1^{GFP} mice were incubated with free sCpG^{5.5} (5 μg/mL) or CNT-sCpG^{5.5} (2.5 μg CNT-5 μg sCpG^{5.5}/mL). The sCpG^{5.5} uptake was visualized by fluorescent microscopy and quantified by flow cytometry. Dot plots demonstrated that CNT-sCpG uptake by BMM was more efficient than update of free sCpG^{5.5} (FIGS. 1A-1D), which was mostly localized to cytoplasmic compartments (FIG. 1E). CNT conjugation also increased sCpG uptake by GL261 gliomas, but not as strongly as in the sCpG uptake by BMM at the same doses of CNT-sCpG^{5.5} or free sCpG^{5.5} (FIGS. 2A-2F, Data were representative of three separate experiments. (n=3 samples/point, ±SEM); *, P<0.05)). When BMM or GL261 gliomas were incubated with the same doses of CNT-sCpG^{5.5} (2.5 μg CNT-5 μg sCpG^{5.5}/mL), sCpG^{5.5} (5 μg/mL) or PBS (control), no significant cytotoxicities were observed (FIGS. 3A and 3B, n=3 samples/point, ±SEM; *, P<0.05, ns: not significant when compared to PBS group). These findings suggested that CNT conjugations enhanced intracellular CpG uptake in vitro. Whether more efficient CpG uptake augmented monocyte activation was examined next in Example 2.

### Example 2

### CNT-Mediated CpG Delivery Potentiated Monocyte Activation In Vitro

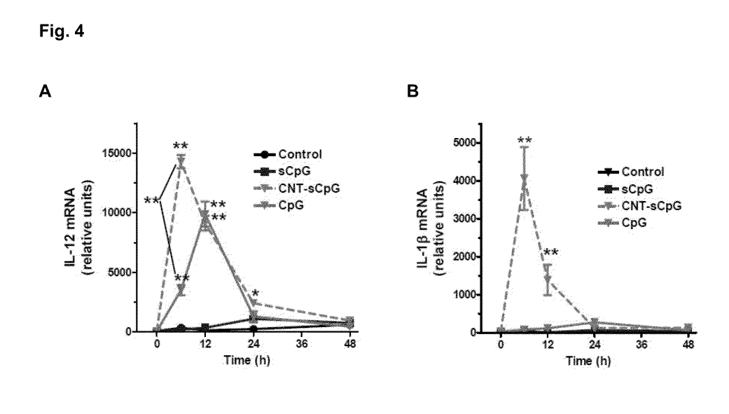

CNT-sCpG (2.5 μg CNT-5 μg CpG/well), sCpG (5 μg/well), or CpG (5 pg/well) were incubated with BMM (5×10⁴ cells/well in 24-well plates) and the cytokine expression was evaluated by qRT-PCR (FIG. 4, n=3 samples/point, ±SEM are shown in the figure; *: P<0.05; and **: P<0.001).

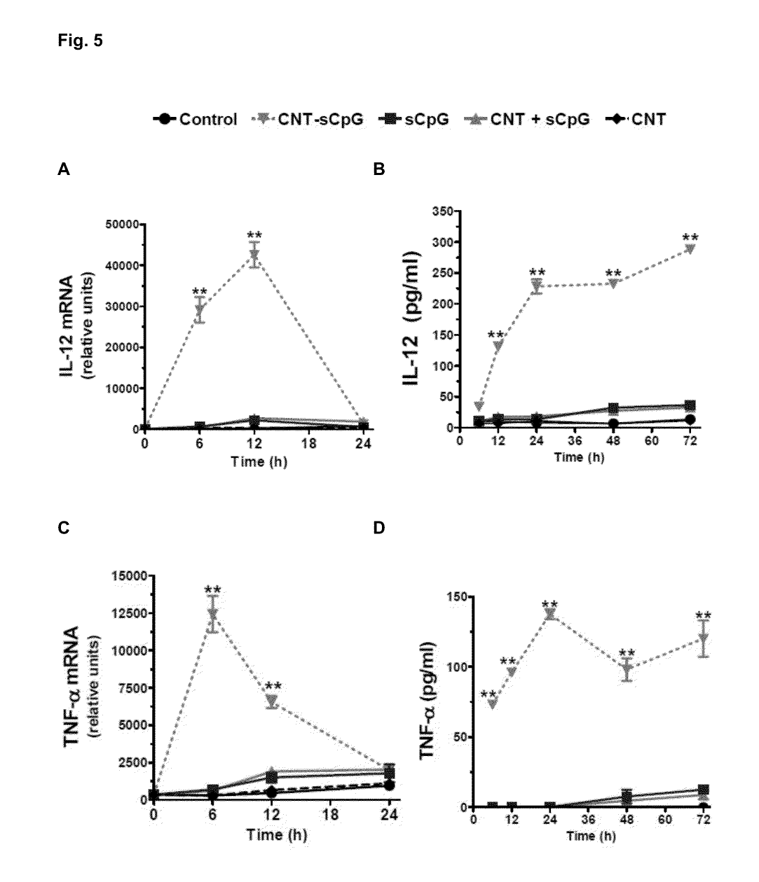

BMM (5×10⁴ cells/well in 24-well plates) were incubated with sCpG (5 pg/well), CNT-sCpG (CNT 2.5 μg-sCpG 5 μg/well), blank CNT (2.5 μg/well), or CNT/sCpG mixture (CNT+sCpG) and cytokine/chemokine expression was evaluated by qRT-PCR and ELISA. The data were representative of two separate experiments (FIG. 5, n=3 samples/point, ±SEM; *: P<0.05; and **: P<0.001).

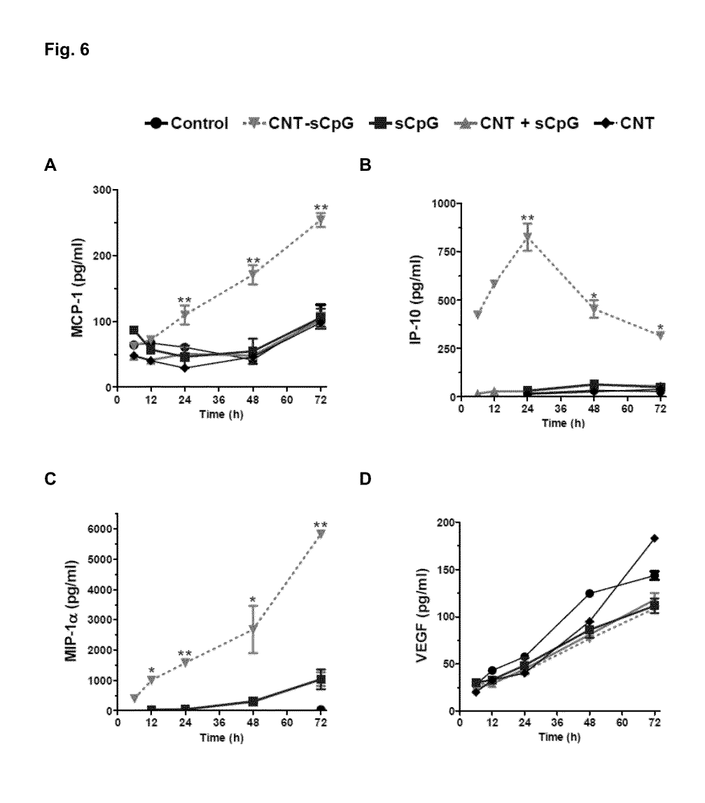

The thiolation process that was employed to link CpG with CNT appeared to suppress CpG pro-inflammatory function (FIG. 4). Nevertheless, when the equivalent doses of sCpG were delivered with CNT-sCpG, a significant upregulation of IL-12 and TNF-α expression was seen (FIG. 5). CNT-sCpG also enhanced release of chemokines, but not VEGF (FIG. 6). Thus, CNT conjugation appeared to not only improve CpG uptake, but also enhanced its proinflammatory function in BMM. The efficacy of CNTs as a potential CpG carrier and immunotherapy agent in i.c. gliomas were tested next in Example 3.

### Example 3

### CNTs Enhanced CpG Uptake by Tumor-Associated Inflammatory Cells

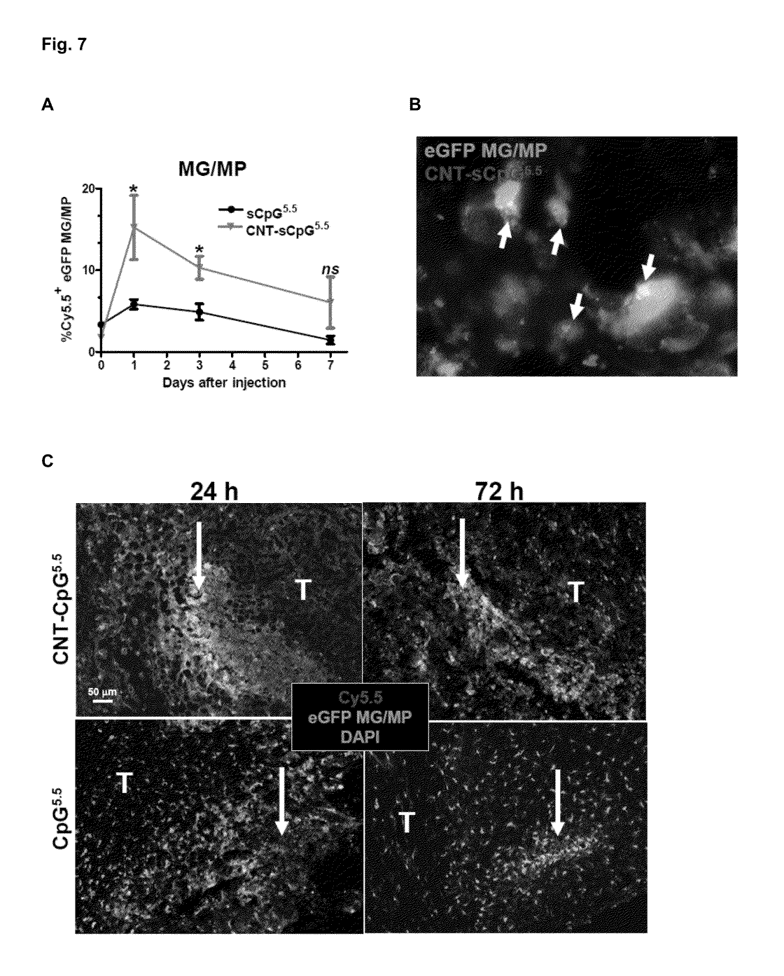

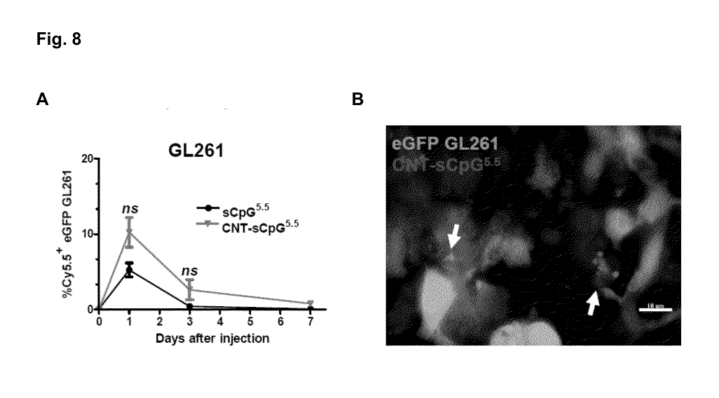

To evaluate CNT-sCpG uptake in vivo, two different i.c. GL261 glioma models were used. To assess uptake by tumor-associated inflammatory cells, GL261 cells were implanted into CX3CR1^{GFP} mice that express eGFP under control of the endogenous Cx3cr1 locus. Although in these transgenic mice eGFP is expressed in MG, MPs, and other myeloid-derived cells, the flow cytometry studies (not shown) have indicated that the majority (more than 70%) of eGFP-expressing cells in this glioma model are MPs (CD11b⁺, CD45^{high}) and MGs (CD11b⁺, CD45^{low}) based on previously-described phenotype characterization (26). In the second model, GL261.egfp cells were i.c. implanted into w.t. mice in order to measure sCpG uptake by tumor cells. Four (GL261 model) or ten days (slower-growing GL261.egfp model) after i.c. tumor implantation, the tumors were injected with CNT-sCpG (2.5 μg CNT/5 μg sCpG/10 μL PBS) or sCpG, (5 μg/10 μL PBS) and tumor GFP cells were sorted and examined for Cy5.5 uptake at various time points (the left panels of FIGS. 7A-7B and 8, n=3 mice/time-point; ±SEM; *, P<0.05; and ns: not significant). Most of the CNT-sCpG (arrows) was seen in the non-GFP-expressing cells (right panels of FIGS. 7A-7B and 8).

Consistent with the in vitro experiments, CNT conjugations enhanced sCpG uptake by tumor-associated inflammatory cells, and to a lesser (non-significant) degree, by gliomas. Interestingly, the representative fluorescent micrographs of brains of the treated mice showed that both sCpG^{5.5} particles and sCpG^{5.5+} MG/MP persisted in the brains of these animals for more than seven days after the initial injection (FIGS. 7A-7C, n=3 mice/time-point; ±SEM; *, P<0.05, T: tumor, ns: not significant). The slower sCpG clearance in CNT-sCpG^{5.5}-injected mice may be due to either the slower clearance of CNT-sCpG by inflammatory cells compared to that of free sCpG, and/or, the robust chemokine release in CNT-sCpG group prevented migration of sCpG^{5.5+} inflammatory cells away from the injection site.

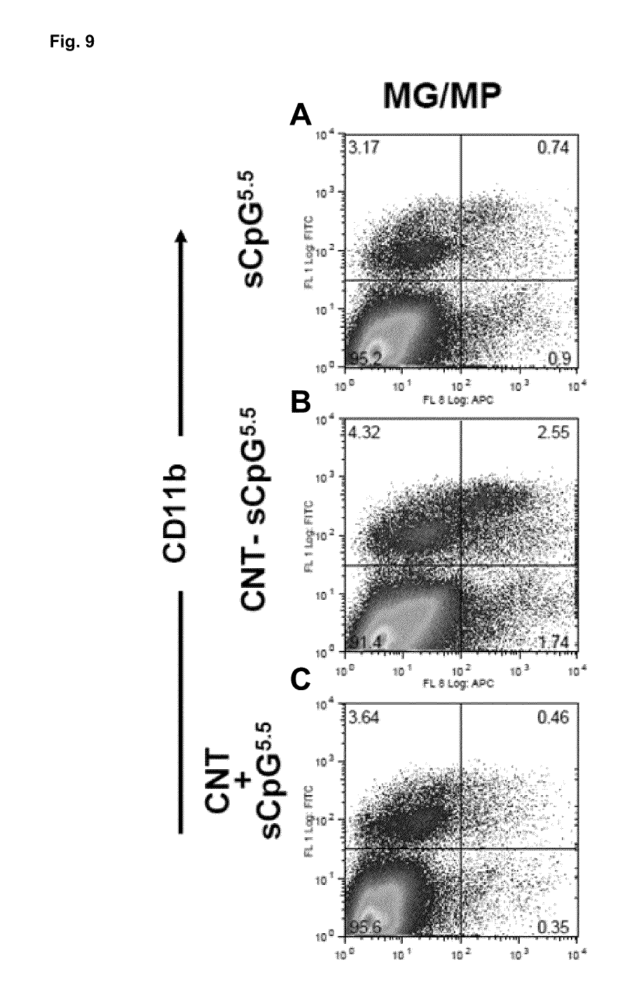

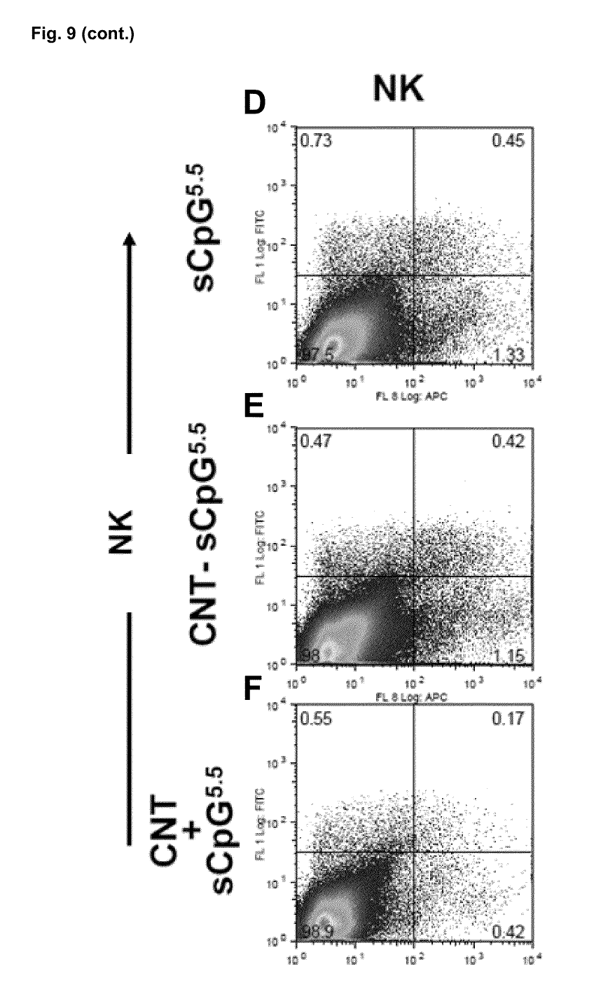

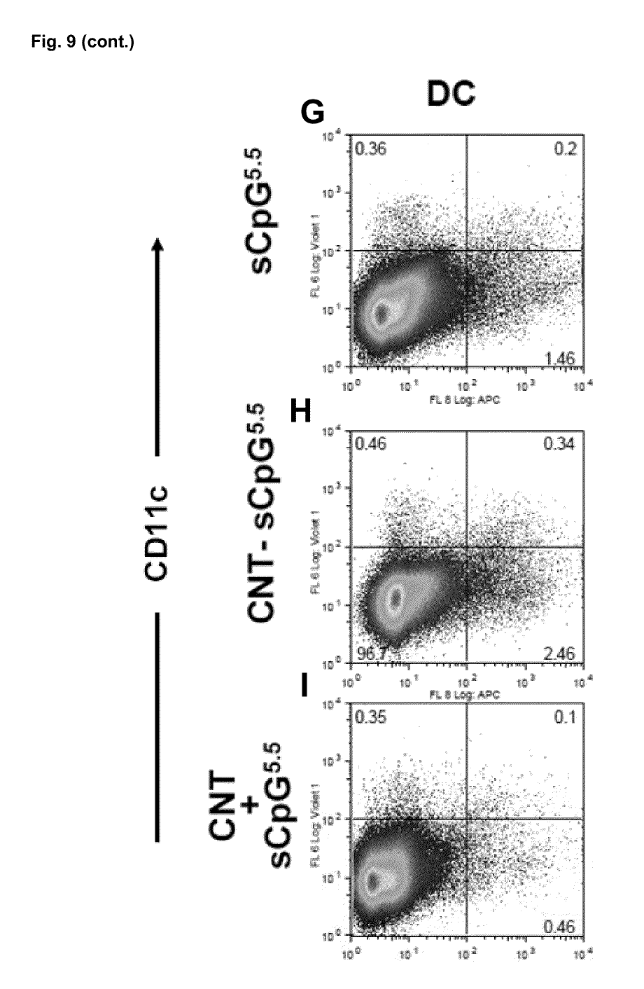

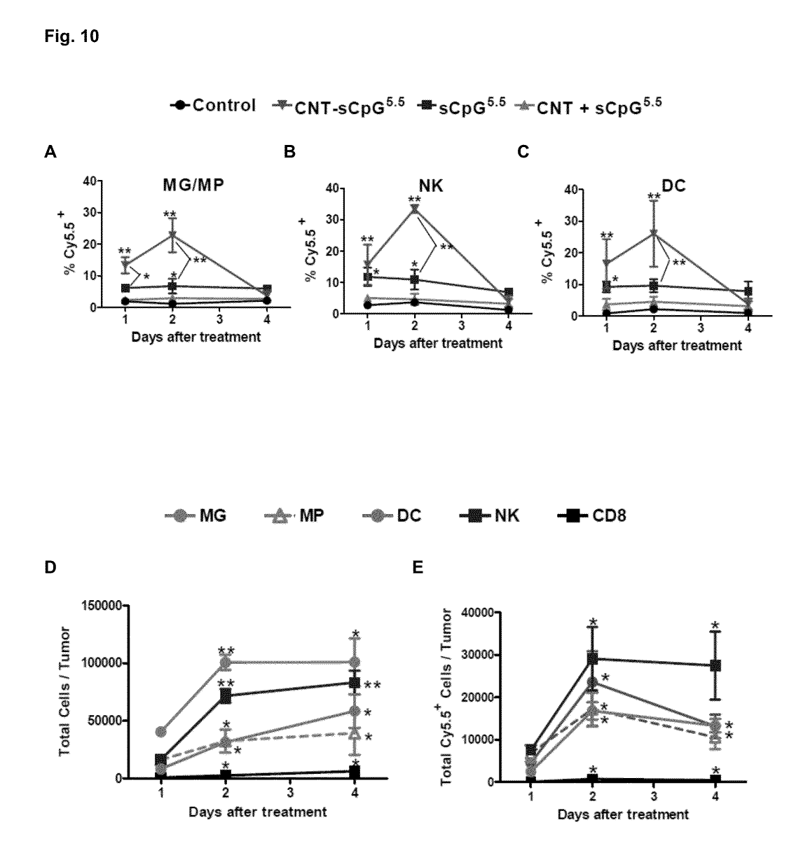

To further evaluate CNT-sCpG⁺ cell types, i.c. GL261 tumors in w.t. mice were injected with PBS (control), CNT-sCpG^{5.5}, free sCpG^{5.5} or CNT/sCpG^{5.5} mixture at the same concentration as in FIG. 5 of Example 2, and the phenotypes of Cy5.5⁺ inflammatory cells were characterized by FACS. The dot plots of tumor-inflammatory cells illustrated stronger CNT-sCpG^{5.5} uptake by tumor-associated microglia and macrophages (MG/MP), NK, and dendritic cells (DC) as compared to free sCpG or CNT/sCpG (FIG. 9). The proportion of CNT-sCpG^{5.5}-positive MG/MP, NK and DC increased within 24 hr of CNT-sCpG injection were determined (FIGS. 10A-10C). The total and CNT-sCpG^{5.5}-positive inflammatory cells in tumors were also measured and determined (FIGS. 10D-10E). Representative data from two experiments were shown in the figures, n=3 mice/time-point ±SEM; *, P<0.05, **, P<0.001 for comparisons to control group in FIGS. 10A-10C and to 24 hr time-point in FIGS. 10D-10E.

As expected, CNT conjugation promoted CpG uptake by MG/MP, NK, and DC cell (but not CD8 cells, not shown) (FIGS. 9 and 10A-10C). Although 20-30% of tumor leukocytes internalized CNT-sCpG^{5.5} particles within two days, the actual proportion of cells that were sCpG^{5.5+} locally was probably much higher because cells from the entire tumor (and not just the injection site) were analyzed in these experiments (FIG. 10A-10C).

Also, to assess which cell types were the prominent carriers of CNT-sCpG, and to check if the decline in sCpG^{5.5+} cells in the CNT-sCpG^{5.5} group at four days was due to the migration of these cells away from the injection site or trafficking of circulating sCpG^{5.5}-negative cells into the tumor, the total number of each cell type for all treatment groups were measured. Neither sCpG nor sCpG/CNT treatments induced a significant change in tumor leukocyte infiltration (not shown). However, in the CNT-sCpG-treated mice, total and Cy5.5⁺ cells rapidly increased within two days of injection before stabilizing (FIGS. 10D-10E). Furthermore, CD11b⁺ cells (i.e. MG and MP) were the most frequent inflammatory cell and CNT-sCpG⁺ cell type within tumors. These findings suggested that 1) CNT delivery enhanced CpG-induced innate immune response to gliomas, and 2) the decline in the proportion of CpG^{5.5+} inflammatory cells was most likely due to both influx of circulating cells into tumors and migration of CNT-sCpG-carrying cells out of the tumor environment. Whether this inflammatory response resulted in tumor rejection was evaluated next in Example 4.

### Example 4

### CNT-sCpG Improved Survival of Glioma-Bearing Mice

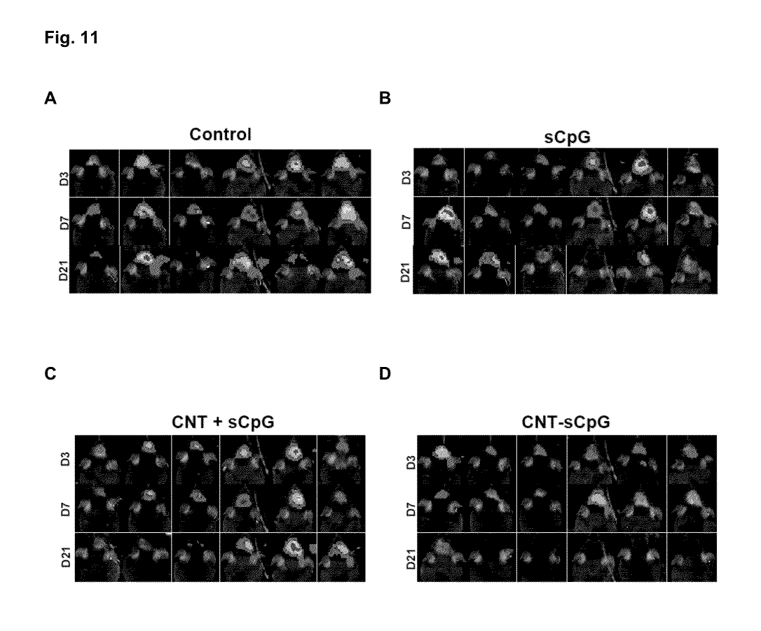

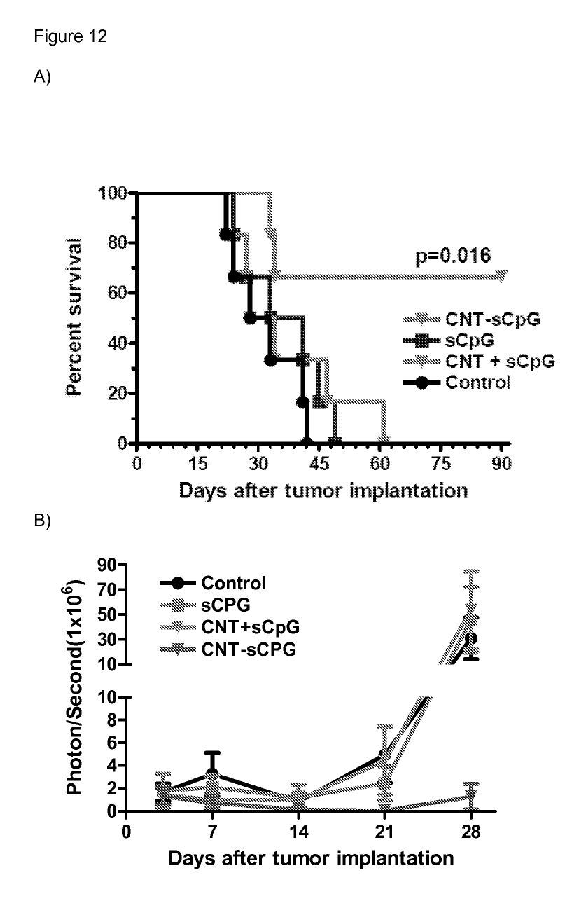

To evaluate CNT-sCpG immunotherapy, mice bearing four day-old i.c. GL261.luc gliomas were given a single intratumoral (i.t.) injection of PBS (control), free sCpG (5 μg/10 μL PBS), CNT+sCpG (2.5 μg CNT+5 μg sCpG/10 μL PBS), or CNT-sCpG (2.5 μg CNT−5 μg sCpG/10 μL PBS). The CNT/sCpG dose combination was selected based on the CNT conjugation capacity of sCpG (discussed above) and the observation indicating CpG to effectively eradicate gliomas at this dose (i.e. 5 μg), albeit after multiple injections (21). Intracranial tumor burden was assessed by biophotonic imaging of mice at 3, 7, and 21 days after tumor injection (FIG. 11). Kaplan-Meier analysis was carried out to assess the survival of mice treated with a single injection of CNT-sCpG (FIG. 12A).

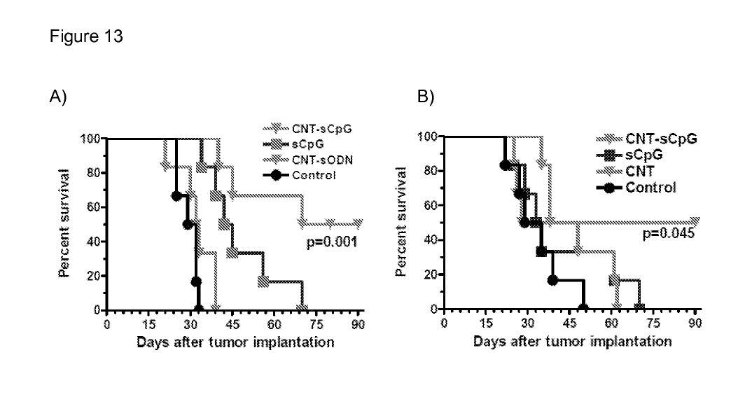

Mice bearing four day-old i.c. GL261.luc gliomas were given a single i.t. injection of PBS (control), free sCpG (5 μg/10 μL PBS), blank CNT (2.5 μg), CNT-sODN (2.5 μg CNT/5 μg sODN/10 μL PBS) or CNT-sCpG (2.5 μg CNT/5 μg CpG/10 μL PBS). Kaplan-Meier analysis was used to assess the survival for the treated mice (FIG. 13). n=6 mice/group.

As seen in the previous in vivo studies (21), a single low-dose injection of free CpG had no anti-tumor effect (FIGS. 11 and 12A). In contrast, CNT-sCpG not only delayed tumor growth, but also cured 50-60% of mice with established gliomas (FIG. 13). Blank CNT by itself, or CNT-sODN, had no effect on tumor growth (FIG. 13). FIG. 12B also showed the effects of CpG, CNT+CpG and CNT-CpG on tumor growth.

The exact mechanism for this improved sCpG therapy with CNT-conjugated delivery is unclear but may have been due to, without being bound to a specific mechanism, 1) higher sCpG uptake by tumor-associated TLR9 inflammatory cells (such as MG/MP), 2) slower intra- or extra-cellular sCpG release and clearance by CNTs (depot effect), and/or 3) migration of CNT-carrying tumor inflammatory cells into lymphoid organs where other immune cells are activated.

### Example 5

### Effects of CNT-sCpG on Tumor-Associated Inflammatory Responses

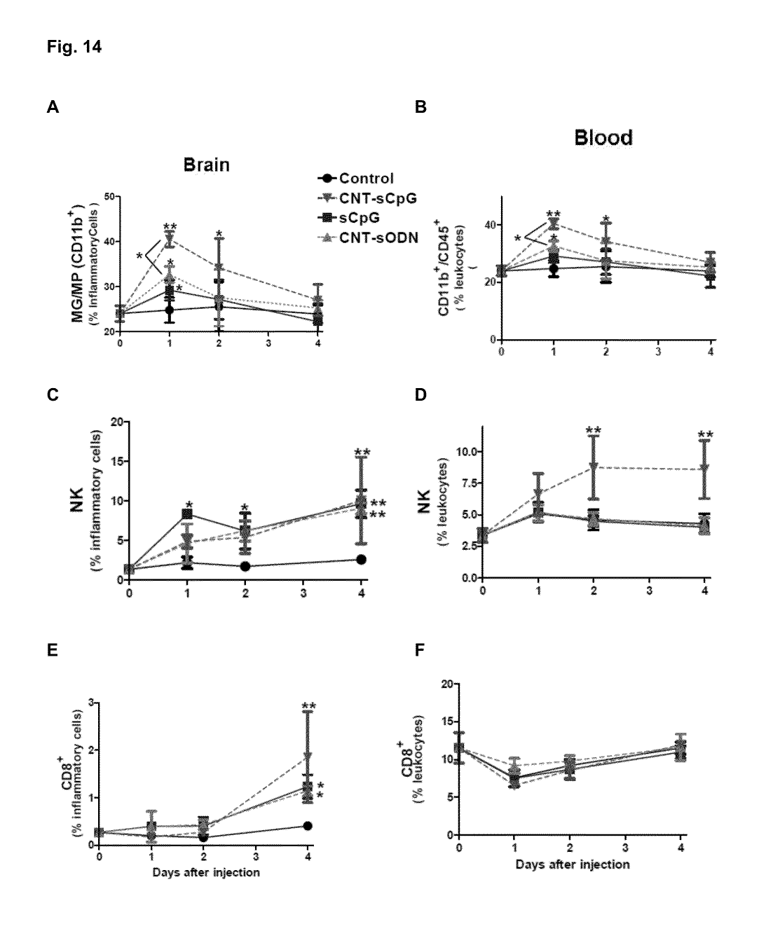

In order to investigate inflammatory cellular responses to CNT-sCpG therapy, mice bearing 4 day-old i.c. tumors were treated with PBS, sCpG, CNT-sCpG or CpG-sODN of the same doses as in Example 4, and the local and systemic inflammatory changes were analyzed by FACS (FIG. 14). Representative data from two experiments were shown, n=3 mice/time-point; ±SEM; *: P<0.05; and **: P<0.001.

Although all agents increased CNS MG/MP, CD8, and NK cell responses, only CNT-sCpG caused a significantly higher infiltration of MG/MP over free sCpG. Furthermore, only CNT-sCpG-treated mice demonstrated a sustained elevation of circulating NK cells. These observations suggested, without being bound to a specific mechanism, that both MG/MP and NK cells played a role in the CNT-sCpG anti-tumor response. To better characterize which cell types were responsible for tumor rejection, leukocyte depletion studies were performed next in Example 6.

### Example 6

### Anti-Glioma Effect of CNT-sCpG was Mediated by Both CD8 and NK Cells

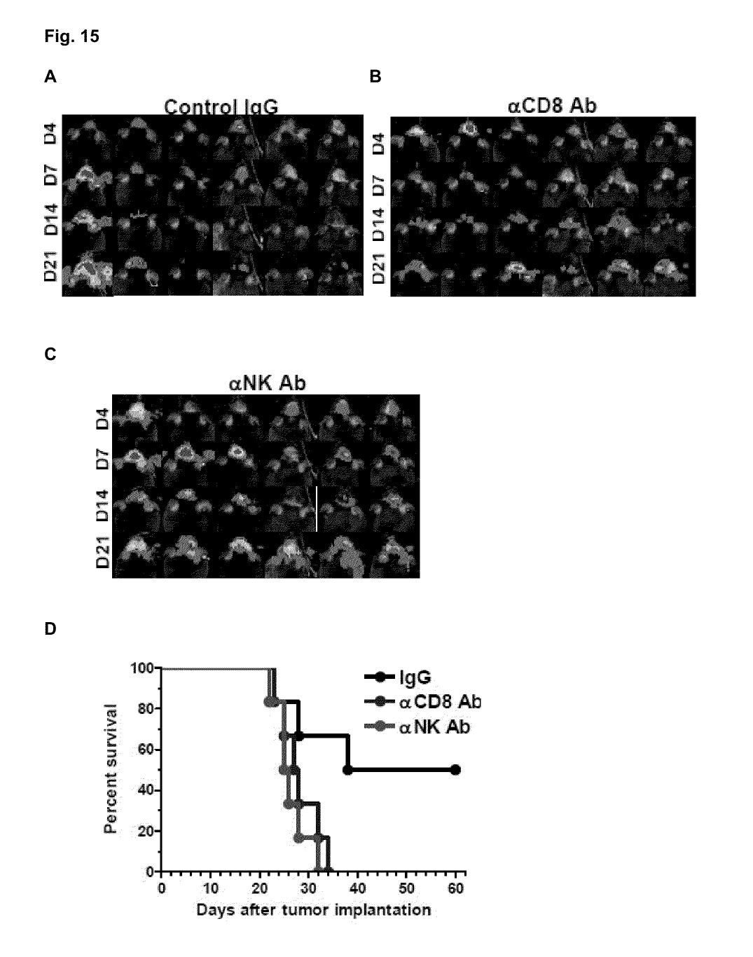

Naïve mice were depleted of CD8 or NK cells by intraperitoneal (i.p.) injection of depleting doses of mAbs specific to CD8 or NK (αCD8 or αNK) or treated with control IgG (200 μg/injection) one day prior to both tumor implantation and CNT-sCpG (2.5 μg CNT/5 μg CpG/10 μL PBS) treatments. Intracranial tumor burden was assessed by biophotonic imaging at day 4, 7, 14, and 21 post tumor implantation (FIGS. 15A-15C). N=6/group. NK and CD8-depleted mice exhibited more rapid tumor growth and lower survival rates (FIG. 15D). The CNT-sCpG anti-tumor effect was completely abrogated by either NK or CD8 depletion (FIG. 15), suggesting that both cell types played a role in the tumor rejections.

### Example 7

### CNT-sCpG-Treated Mice Developed Immunity Against Glioma Rechallenge

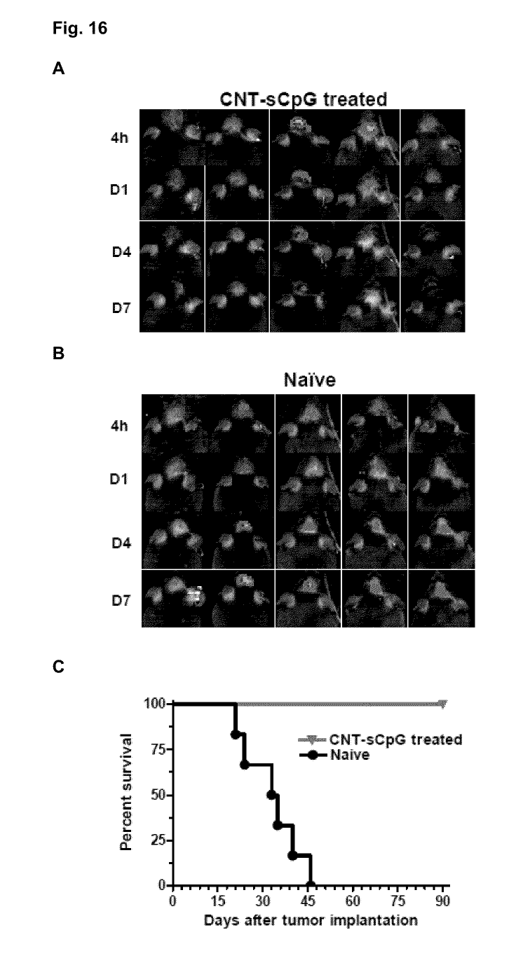

To determine if CNT-sCpG treatment induced immunity against gliomas, CNT-sCpG-treated GL261-bearing mice that had survived for at least three months, along with normal naïve mice, were rechallenged with an i.c. injection of GL261 glioma(1×10⁵). Intracranial tumor burden was assessed by biophotonic imaging at 4 h, days 1, 4, and 7 after the tumor implantations (FIG. 16). n=5 mice/group. The results showed that the “cured” mice developed full immunity against GL261 rechallenge (FIG. 16).

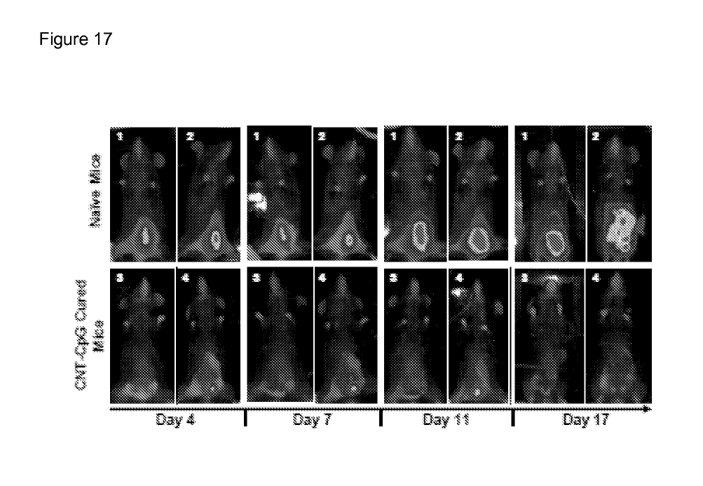

FIG. 17 showed induction of systemic anti-glioma immunity with intracranial (i.c.) CNT-CpG therapy. Naive mice (FIGS. 17, 1 and 2) and mice bearing i.c. GL261 gliomas (FIGS. 17, 3 and 4) that had survived i.c. CNT-CpG therapy were rechallenged with tumor injection into the flank. Tumor growth was assessed by BLI.

### Example 8

### Examination of CNT-CpG Activity Using an In Vitro NF-κB Assay

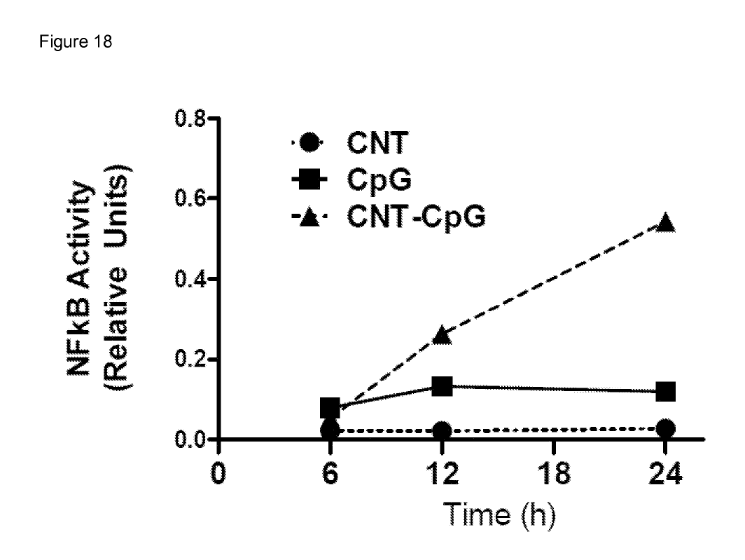

Before animal experiments, CNT-CpG activity was examined using an in vitro NF-κB assay as described supra (FIG. 18). CNT-CpG preparations that led to at least a doubling of NF-κB activity, as compared to an equivalent dose of free CpG at 24 hours, were selected for in vivo and in vitro experiments in Examples 9˜12.

### Example 9

### CNT-CpG Antitumor Response (In Vivo)

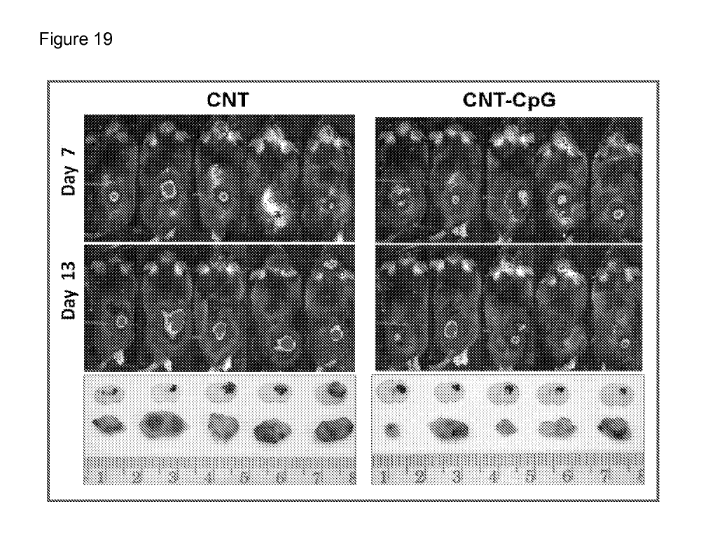

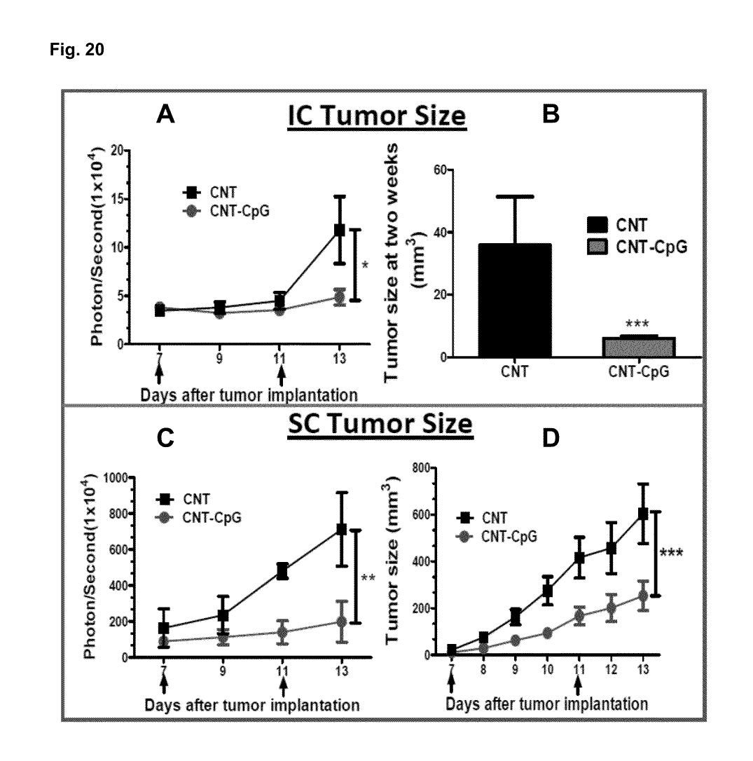

To confirm whether i.c. CNT-CpG elicited a systemic antitumor response, mice bearing both i.c. and s.c. B16F10-luc melanomas were treated twice (7 and 11 days (arrows) after initial tumor implantations) with i.c. CNT-CpG (2.5 μg CNT/5 μg CpG/10 μL, ) or i.c. CNT (2.5 μg/10 μL, ▪) that were functionalized only with PL-PEG (FIG. 19). Tumor growth was evaluated by Xenogen (FIGS. 19 and 20A-20D) and direct tumor size measurements with calipers (FIGS. 20A-20D). The data were representative of two separate experiments. n=5 mice/group; and *: P<0.05.

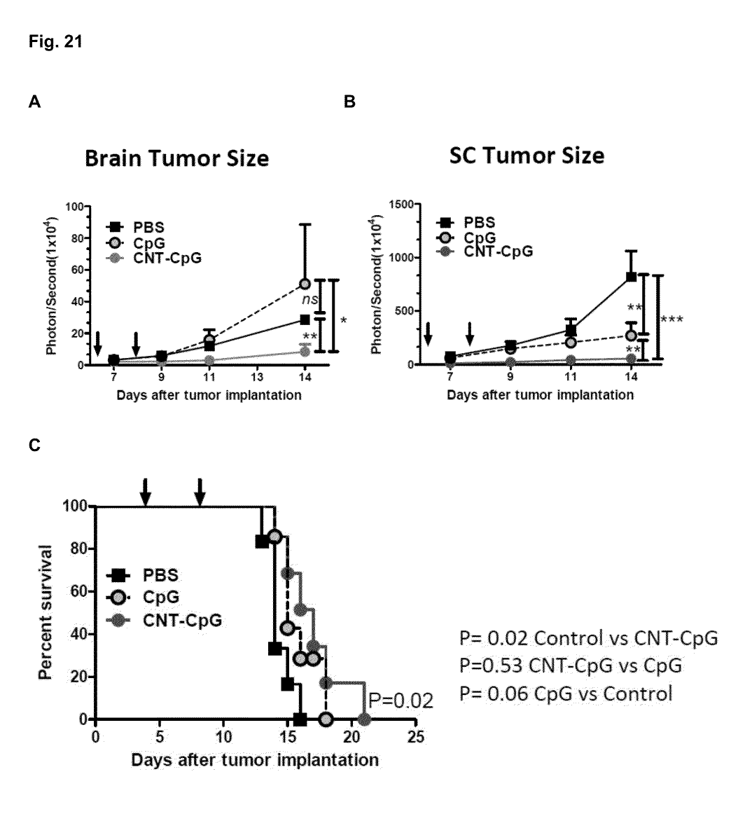

When administered i.c., CNT-CpG (but not blank CNT) inhibited the growth of not only i.c., but also, s.c. melanomas (FIGS. 19 and 20). To confirm whether this anti-tumor effects were specific for CNT conjugated delivery, a group of mice were treated with i.c. free CpG (5 μg/10μL) in a similar experiment as described supra in this example, and the tumor growth was evaluated by brain and s.c. tumor sizes (FIGS. 21A-21B) and the survival rate (FIG. 21C). At these low doses, i.c. CpG showed no effect on i.c. tumors, but inhibited s.c. tumor growth, albeit not as much as CNT-CpG (FIG. 21). The results showed that that low doses of i.c. CNT-CpG induced both a local and a systemic anti-tumor response.

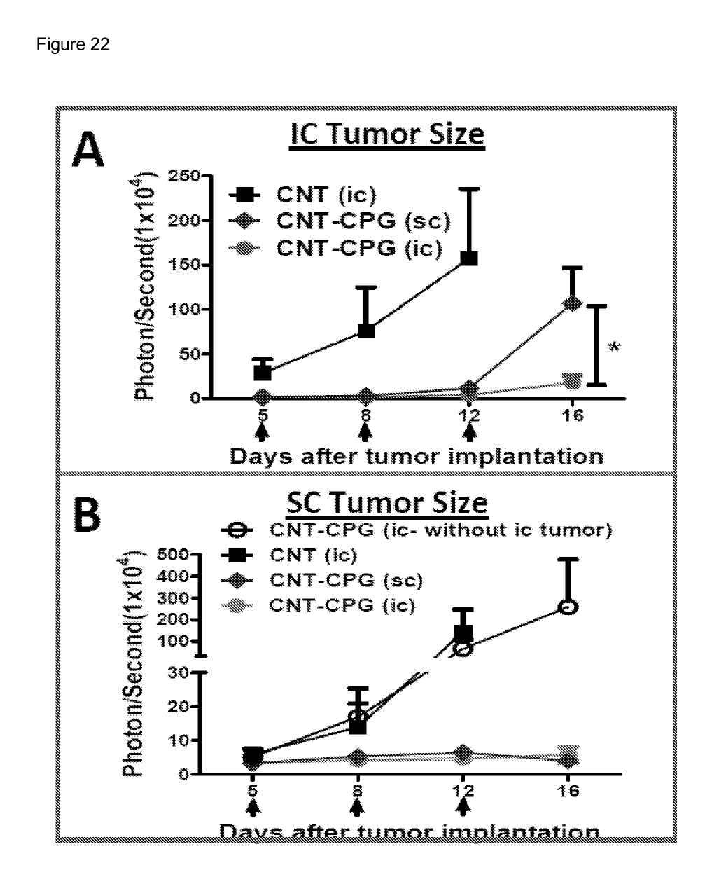

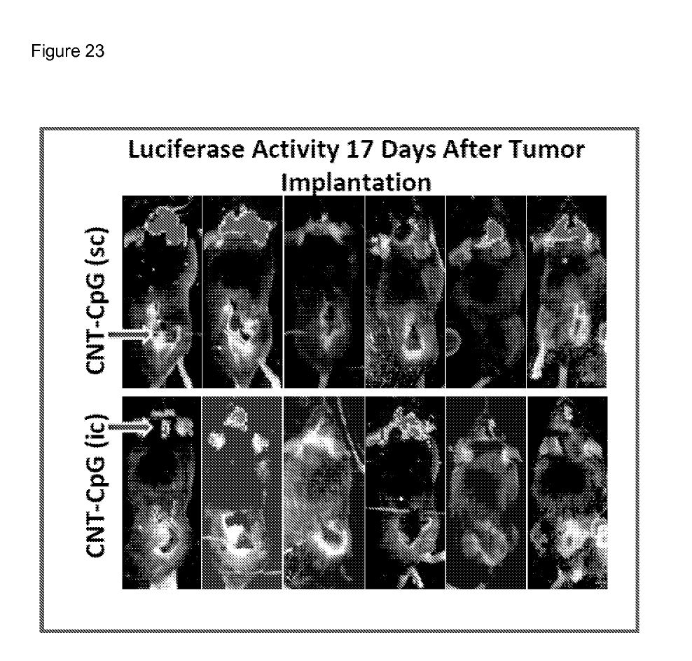

To evaluate the impact of injection site on CNT-CpG efficacy, a similar experiment as described supra in this example was performed except that the animals were treated with either s.c. or i.c. CNT-CpG three injections (5, 8, and 12 days) after tumor implantation (FIG. 22, arrows). Tumor growth was evaluated by Xenogen imaging (e.g. as shown in FIG. 23). Arrows in FIG. 23 indicated site of CNT-CpG injections. n=6 mice/group; and *: p<0.05.

Similar to the previous experiments, i.c. CNT-CpG (but not blank CNT) inhibited the growth of both i.c. and s.c. melanomas (FIGS. 22A and 22B, ), and was more effective than s.c. CNT-CpG at inhibiting the growth of both i.c. and s.c tumors. However, development of this systemic immune response required tumor presence in the CNS, as mice that lacked i.c. melanomas did not exhibit systemic antitumor activity (FIG. 22B). While s.c. CNT-CpG inhibited s.c. tumors (FIGS. 22A and 22B, ), it was not as effective as i.c. CNT-CpG in abrogating i.c. tumor growth (FIG. 22A, ♦, FIG. 23). These results suggested that, without being bound to any specific mechanism, either CNS-specific micro-environmental factors induced a stronger effector response to CNT-CpG that was capable of inhibiting tumor growth irrespective of its location, or i.c. CNT-CpG enhanced trafficking of cytotoxic leukocytes into the brain.

Because CNT-CpG biodistribution directly affected its function, CNT-CpG clearance in both i.c. and s.c. tumors were compared in Example 10.

### Example 10

### CNT-CpG Biodistribution

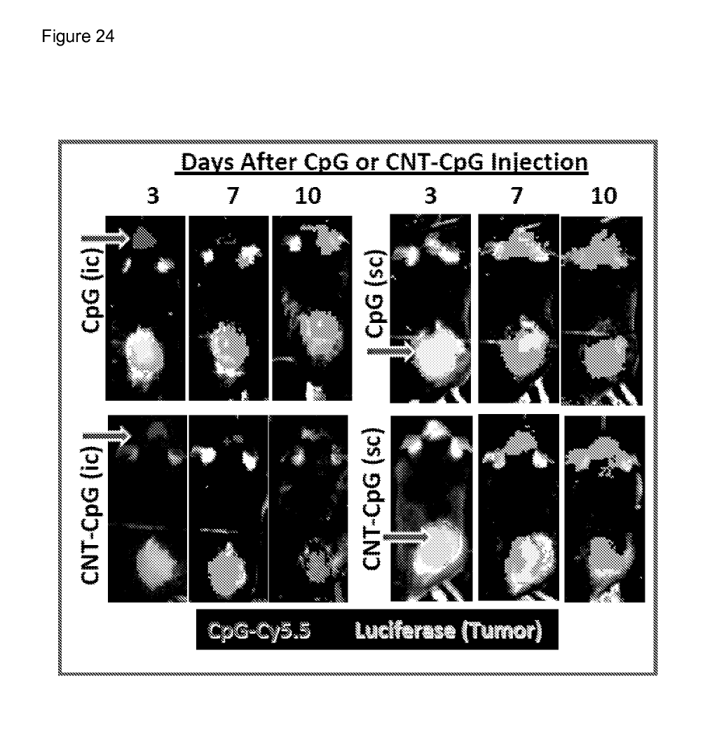

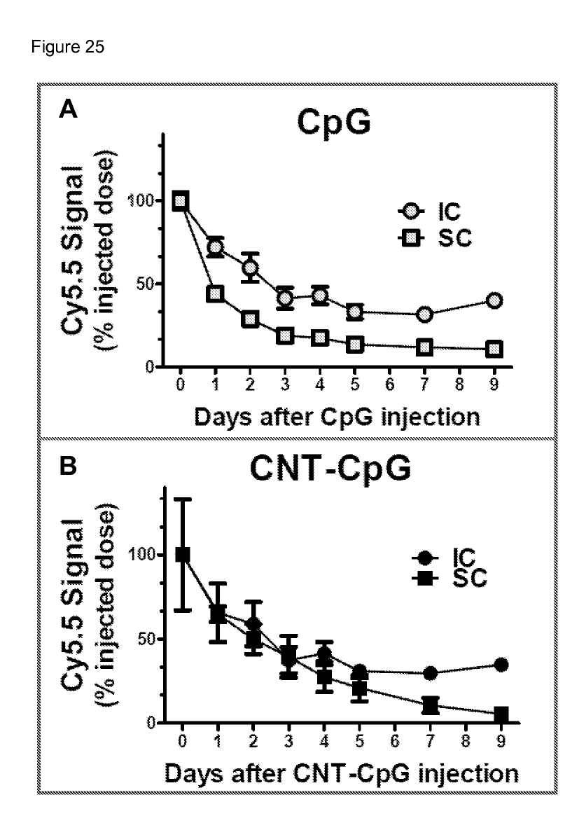

To study the potential differences in CNT-CpG uptake and retention in tumors, mice bearing both i.c. and s.c. B16-luc melaomas (green) were injected i.t. with Cy5.5-labeled (red) CpG^{5.5} or CNT-CpG^{5.5} into the four-day old i.c. or s.c. tumors (indicated by arrows, FIG. 24). Tumor growth and Cy5.5 signal were then monitored (FIG. 24), and measured (FIG. 25) by Xenogen imaging.

Representative mice from each group were shown in FIG. 24, which demonstrated CNT-CpG and CpG clearance in relation to tumor growth. Both CNT-CpG^{5.5} and free CpG^{5.5} appeared to clear slower from i.c. than from s.c. tumors (FIG. 25). Although initial CNT-CpG clearances were similar in i.c. and s.c. tumors, some Cy5.5 signal was still detectable in the i.c. but not in s.c. melanomas after a week (FIG. 25). Furthermore, CpG clearance from i.c. tumors appeared to be independent of CNT delivery as Cy5.5 signal-drop was similar in animals injected with CpG^{5.5} and CNT-CpG^{5.5}, respectively.

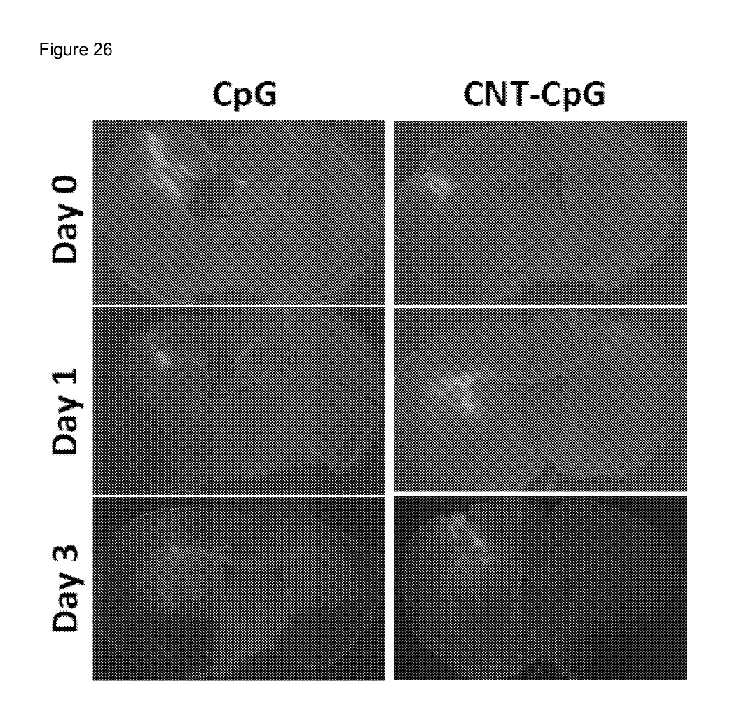

In another experiment similar to the experiment described supra in this example, the brains were harvested, sectioned and imaged with fluorescent microscopy (FIG. 26). The local distribution of free CpG to CNT-CpG in i.c. melanomas were compared (FIG. 26). Within a few days, the free CpG appeared to diffuse away from the injection site, while CNT-CpG appeared to disperse around the tumor (FIG. 26).

Without being bound to a specific mechanism, inflammatory cells (e.g. MG, MP, and NK cells) appeared to be the main carriers of CNT-CpG particles in experimental gliomas (see, Example 3, FIGS. 9 and 10). Thus, even though the overall CpG^{5.5} signal dissipation was similar to that of CNT-CpG^{5.5} (FIGS. 25A and 25B), their biodistributions around the i.c. tumors were different possibly due to the more efficient uptake of CNT-CpG by tumor-associated leukocytes and their migrations around the tumor periphery.

### Example 11

### Role of Tumor Microenvironment on CNT-CpG Inflammatory Responses

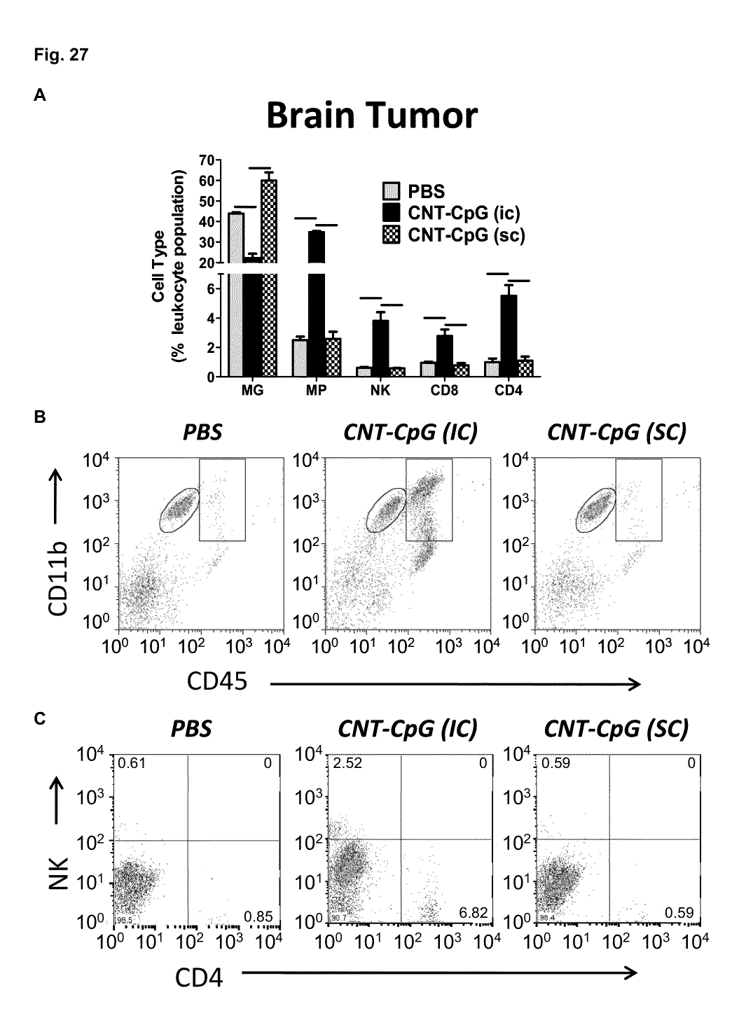

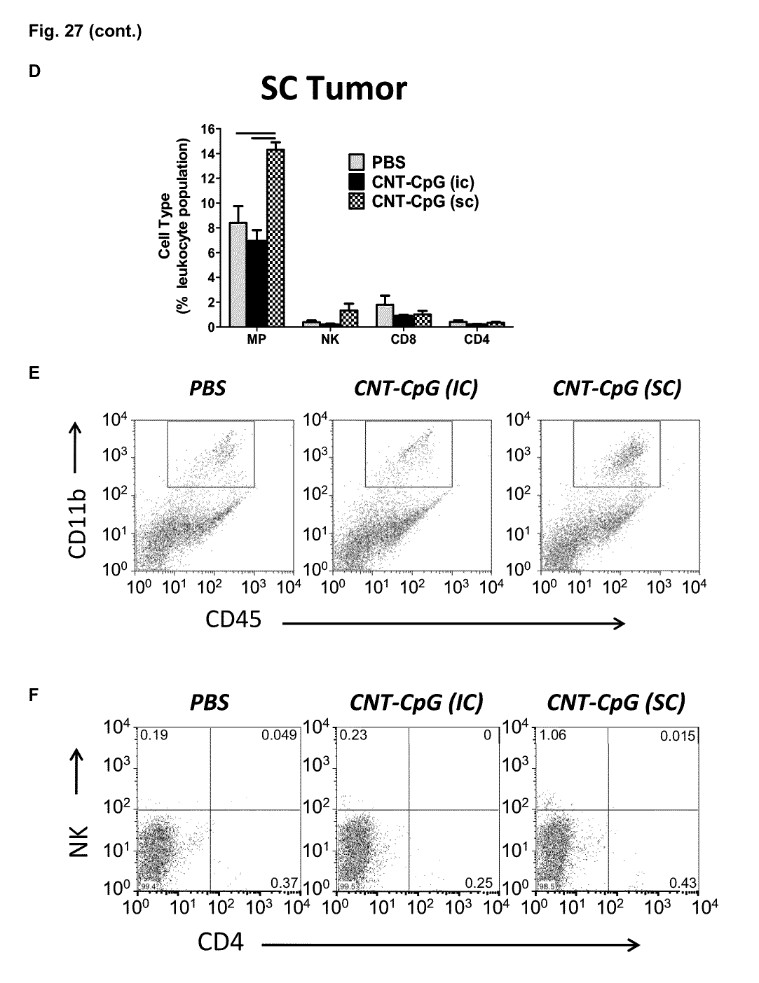

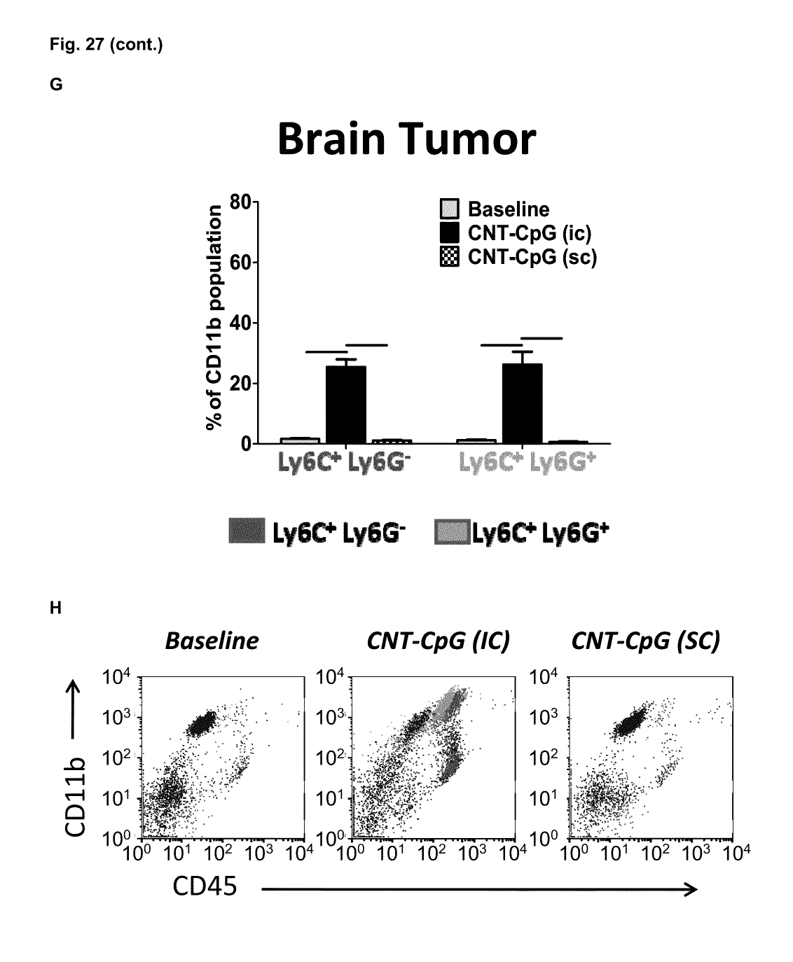

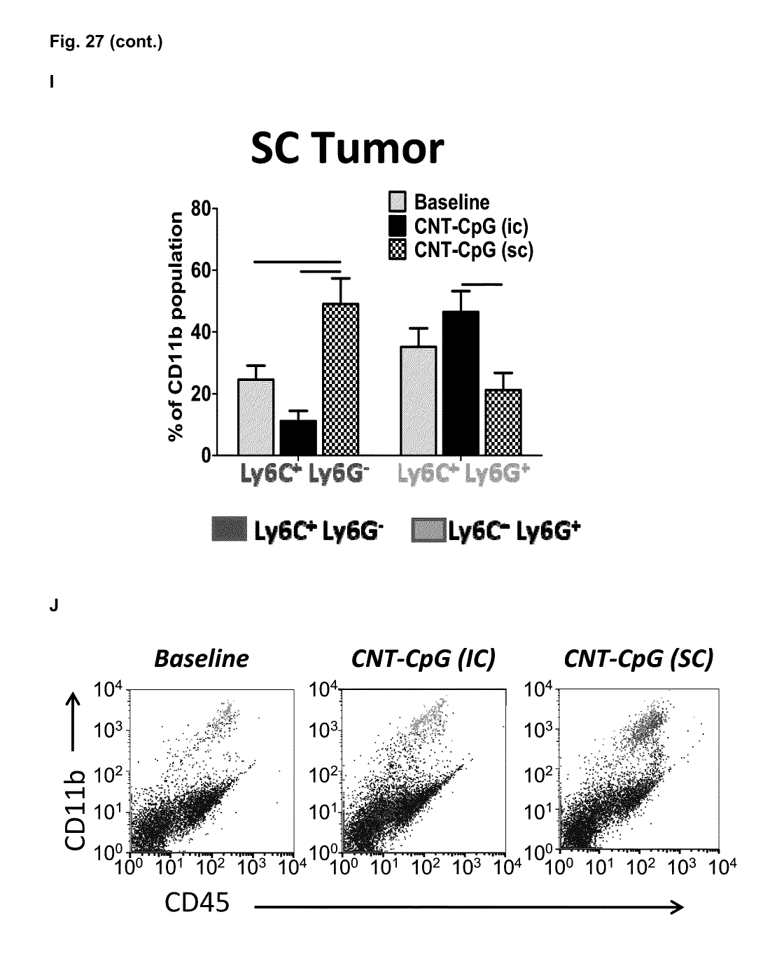

In order to investigate the cellular responses to the CNT-sCpG therapies, mice bearing 4 day-old i.c. and s.c. B16F10 melanomas received i.t. treatment of PBS or CNT-CpG (2.5 μg CNT/5 μg CpG), and the tumors were analyzed by FACS after 24 hours (FIG. 27) or 48 hours after treatment (Data not shown). The proportions of tumor inflammatory cells were quantified by flow cytometry. N=4 mice/group; and the bars and ** represented p<0.05. The intracranial CNT-CpG increased infiltrating MP (CD45hi, CD11b+), NK, CD8, and CD4 cells in i.c. tumors, but had no effect on inflammatory cell infiltration in the non-treated s.c. tumors in the same animals (FIGS. 27D-27F). MG (CD45low, CD11b+) as a proportion of total leukocytes decreased in i.c. tumors after CNT-CpG treatment as a result of influx of other inflammatory cells (FIGS. 27A-27C). In contrast to the i.c. injections, s.c. CNT-CpG promoted MP (CD11b+) infiltration without significantly affecting NK, CD8, or CD4 influx (FIGS. 27D-27F). Similar results were observed at 48 hours after treatment (data not shown). Because CD11b⁺ cells were the most frequent leukocytes, and because these cells comprise a heterogeneous cell population, the impact of tumor micro-environment on CD11b+ phenotypes following CNT-CpG therapy was characterized (FIGS. 27G-27J). At baseline, the proportion of infiltrating MPs with myeloid-derived suppressive cell phenotype (MDSC, Gr1⁺) was very low in the i.c. tumors (FIGS. 27G-27H). After the CNT-CpG injections, however, both Ly6C+ and Ly6G+ cells equally increased in the injected i.c. tumors (FIG. 27G). In the s.c. tumors, however, Gr1+ cells were more prevalent at baseline and significantly increased after the CNT-CpG treatment (FIGS. 271-27J). The proportion of monocytic MDSC (M-MDSC; Ly6C^{high}) was higher than granulocytic MDSC (G-MDSC, Ly6G⁺ Ly6G^{low}) in the CNT-CpG-treated s.c. tumors. Although both cell types were considered to be MDSCs, Ly6C cells have a higher suppressive activity in mice.

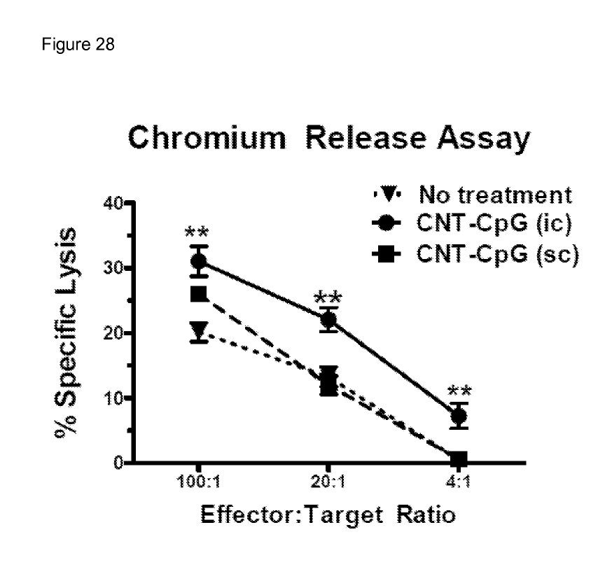

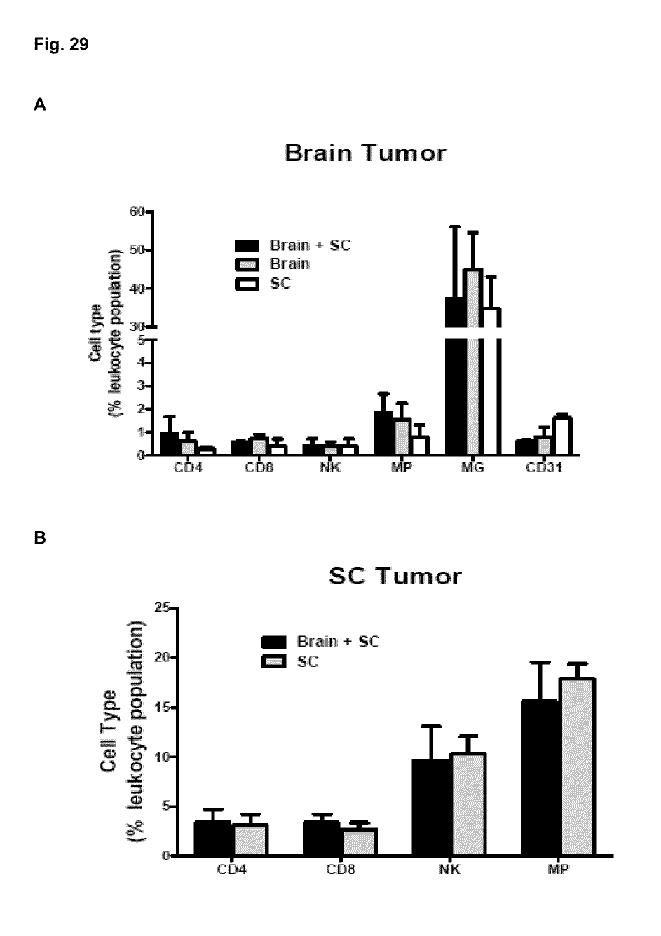

To confirm whether the local cellular responses to CNT-CpG correlated with the systemic antitumor activities, splenocytes of treated animals in the previous experiment were harvested and tested for tumor cytotoxicity. i.t. CNT-CpG elicited a stronger ex vivo anti-tumor response when injected into i.c. melanomas than into s.c. tumors (FIG. 28). Similar observations were made when CpG was tested without CNT delivery (FIG. 29) suggesting that CNS micro-environment factors may have indeed enhanced CNT-CpG (and CpG) antitumor responses. Because CpG function depended on the expression of its receptor, TLR9 expression in melanomas in either location was evaluated in Example 12.

### Example 12

### TLR9 Expression

CD11b+ cells (MG and MPs) were shown to be prominent carriers of CNT-CpG in experimental gliomas (e.g. Example 3, FIGS. 9 and 10). To further characterize micro-environmental differences that accounted for stronger cytotoxic response in i.c. CNT-CpG-treated mice, the expression of TLR9 in tumor-associated leukocytes was studied.

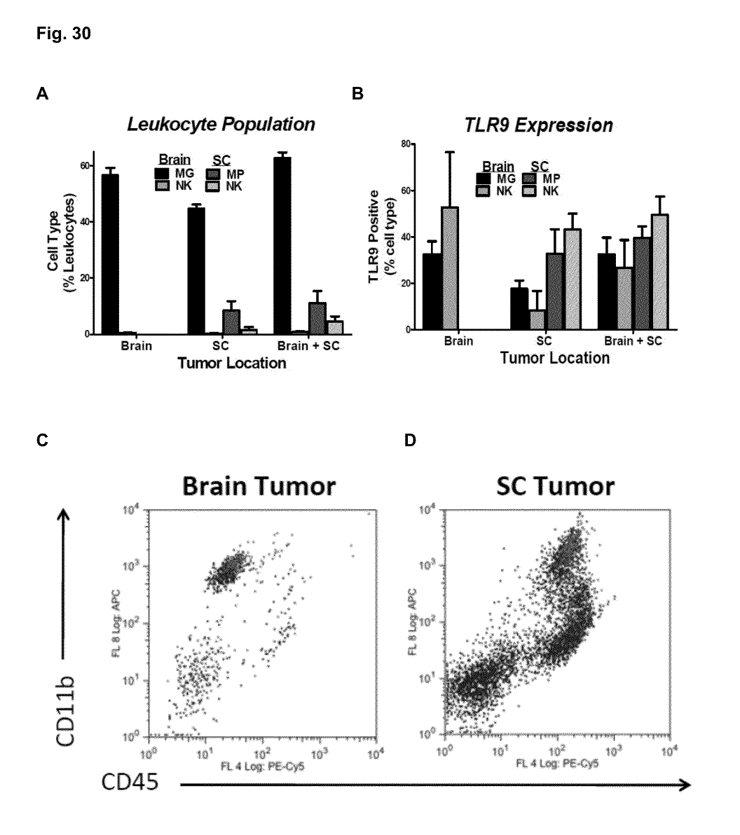

Mice bearing seven-day old i.c., s.c., or both i.c. and s.c. tumors were examined for TLR9 expression (FIG. 30). Microglia were the most common inflammatory cells in newly-implanted i.c. tumors (50-60%) and accounted for most TLR9-positive cells. Infiltrating NK and CD11c cells (not shown), which accounted for less than 10% of tumor leukocytes, also expressed TLR9. In s.c. melanomas, MP, NK and CD11c cells accounted for less than 10% of tumor-associated inflammatory cells but equally expressed TLR9. Concurrent growth of i.c. and s.c. tumors in the same animal did not significantly influence TLR9 expression at this early stage. These findings suggested that presence of TLR9-positive MG in brain tumors may have played a role in CNT-CpG response in i.c. tumors.

### Discussion

Immunotherapy is an attractive treatment modality for immunogenic tumors such as melanomas. Although various approaches for melanoma immunotherapy are been pursued with mixed results, recent findings from a phase III randomized trial of CTLA-1 blocking agent has validated the efficacy of this approach. One limitation for effective melanoma immunotherapy, however, may be the high frequency of brain metastasis which may occur in 10-30% of patients. Although activated T cells can penetrate intracranial tumors, clinical studies have demonstrated less than 50% of CNS metastasis to respond to systemic adoptive immunotherapies in melanoma patients. Thus, there is a need for improved targeting of metastatic brain tumor. While examining the role of CpG immunotherapy with nanoparticles, it was noted that a systemic antitumor response in experimental gliomas (Example 7). Furthermore, i.c. CNT-CpG not only inhibited the growth of brain melanomas, but also had a robust remote inhibitory effect in untreated s.c. tumors (Examples 9-12). These observations, which were unexpected, imply that i.c. CNT-CpG therapy may be applied to the treatment of not only gliomas, but also metastatic brain tumors.

Conjugation of CNT to CpG enhanced CpG immune responses in glioma models. Several features may be unique for CNT-CpG immunotherapy in the brain. First, biodistribution of CNT-CpG and CpG appeared to be different in i.c. tumors than s.c. melanomas. Whereas all of CNT-CpG (and CpG) was cleared from s.c. tumors, some CpG was retained even a week after i.c. injections. Second, CNT-CpG distribution around tumors was different than free CpG. Local MG actively uptook CNT and CpG complexes and since these cells did not migrate out of CNS, their retention and distribution around i.c. tumors may have accounted for a stronger antitumor response when injected into i.c. tumors.

The value of IT i.c. vaccine approaches to enhance systemic therapies has been suggested before but none of these studies evaluated the utility of this approach for systemic immune.

Human relevance: There has been a disparity in CpG immunotherapy results between mice and humans. Initial therapies in mice have shown excellent results, but these findings could not be duplicated in human. This disparately has been attributed to variation in TLR expression. In mice a number of cells (NK, etc.) express high levels of TLR9, but in humans, most TLR expression is localized to DC cells. TLR9, however, has also been shown to express by MG in humans, thus the strategy described herein may still be applicable to human trials. In humans, prDC are most prevalent TLR9-expressing cells in peripheral circulation.

Toxicity has been raised as a potential limitation of CNT biomedical application. While CNTs can be biodegraded through enzymatic catalysis (16), pristine (non-modified/non-functionalized) CNTs have been shown to persist for several months (12) raising concerns of potential negative health effects. fCNTs studied herein were non toxic after i.c. injections in these short-term experiments, thereby supporting their utilization in cancer immunotherapy.

In summary, CNT delivery system was shown to significantly enhance CpG immunotherapy, eradicate i.c. gliomas at low doses, and induce immunity against tumor rechallenge without inducing toxicity. Similar method may be used for CNT-based treatments for malignant brain tumors.

Furthermore, a person of ordinary skill in the art would know that other types/combinations of CNTs (e.g. other SWCNTs, double-walled CNTs, multi-walled CNTs, and combinations thereof) and/or other NANO described herein (e.g. gold, iron, silica, organic polymers, or carbon nanomaterials (e.g. fullerenes, rapheme, nanohorns, nanodiamond, etc.)) may also be used to provide other NANO delivery systems that are similar to the CNT delivery system disclosed herein, and can be used in the methods disclosed herein.

### Example 13

### Preparation of a CNT-CpG-II (e.g. a Type II SWCNT-CpG (SWCNT-CpG-II))

As used in examples 13-17, unless otherwise specified, SWCNT/CpG, SWCNT-CpG, CNT/CpG, CNT-CpG and CNT or SWCNT CpG conjugates are type II NANO-CpG.

1,2-distearoyl-sn-glycero-3-phosphoethanolamine-N-[amino(polyethylene glycol)-2000] (ammonium salt) (Lipid-PEG) was purchased from Avanti Polar Lipids, Inc. Dithiotheritol (DTT) was purchased from VWR. Dithiolated CpG oligonucleotides (5-HO-C6-SS-C6-TAAACGTTATAACGTTATGACGTCAT-3, SEQ ID NO: 15) (RSS-CpG) and Cy5.5 labeled CpG oligonucleotides (5-HO-C6-SS-C6-TAAACGTTATAACGTTATGACGTCAT-C6-Cy5.5-3′, SEQ ID NO: 16) (RSS-CpG-Cy5.5) were provided by the DNA core facility at Beckman Research Institute at City of Hope. Sulfosuccinimidyl 6-(3′-[2-pyridyldithio]-propionamido)hexanoate (Sulfo-LC-SPDP) was purchased from ProteoChem.

Preparation of Lipid-PEG-LC-SPDP-CpG

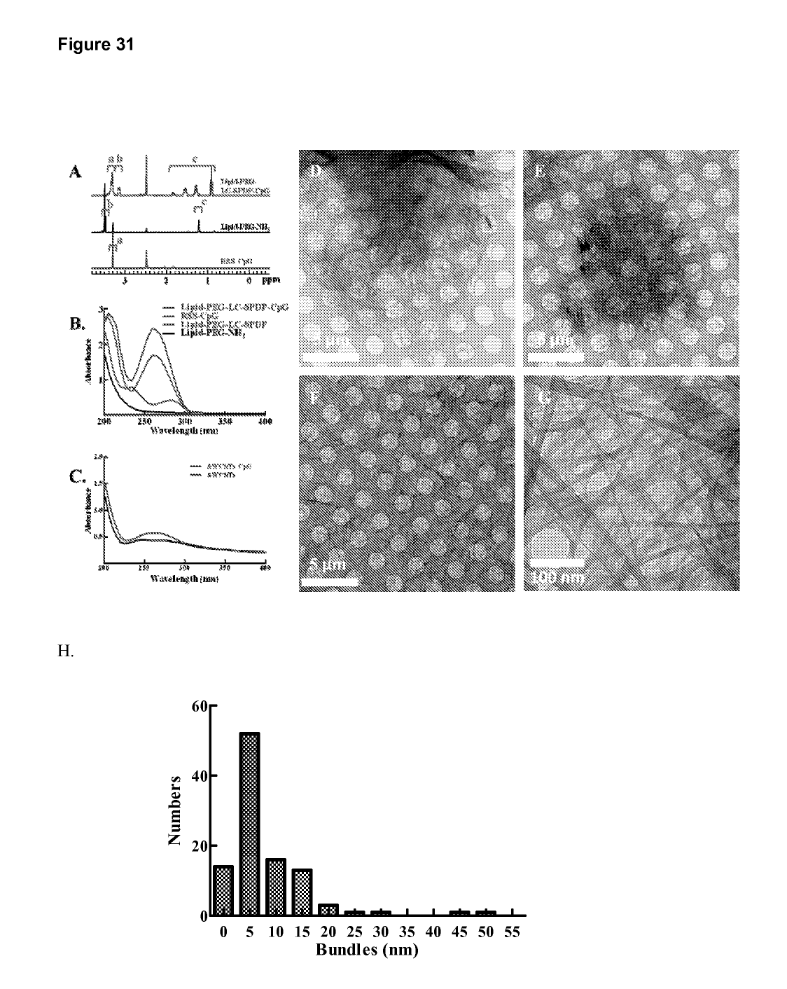

First, 337 mg Lipid-PEG-NH₂ and 52.7 mg Sulfo-LC-SPDP were added to 10 mL distilled water and stirred for 2 hours at room temperature to form Lipid-PEG-LC-SPDP. In the mean time, 50 mg RSS-CpG and 5 mL of 10 mM DTT were added into 15 mL distilled water and stirred for 2 hours at room temperature. DTT treated RSS-CpG was then purified using nap-25 column (GE health care). Concentration of the freshly prepared HS-CpG was determined with NanoDrop 1000 Spectrophotometer (Thermo Scientific). Then, excess Lipid-PEG-LC-SPDP was added to the freshly prepared HS-CpG. The reaction was kept at 60° C. for at least 2 hours. The UV absorbance at 345 nm for the byproduct free 2-mercaptopyridine was monitored by removing 10 μL aliquots from the reaction. When the intensity of this peak stopped increasing the reaction was judged to be complete. To purify the CpG containing polymer, isopropanol was added (by volume, 3× the volume of the solution) and the resulting mixture was stored at −20° C. overnight, then centrifuged at 15,000 rpm for 45 minutes. The obtained pellet was collected and redissolved in water, dialyzed to remove isopropanol, and lyophilized. The proton NMR (¹H NMR) spectra was obtained on a VARIAN NMR system/400-MR using deuterated dimethyl sulfoxide as solvents (FIG. 31A).

Ultrasonication

All ultrasonication in this example was performed using a QSONICA Sonicator Q700 (QSONICA, Newtown, Conn., USA) equipped with a cup horn cooled with running water from the sink. Samples were loaded in 2.0 mL eppendorfs. The sonicator was set at amplitude 80 with pulse-on for 15 seconds followed by pulse-off for 15 seconds to avoid overheating.

Preparation of SWCNT-CpG

First, Lipid-PEG-LC-SPDP-CpG prepared above was dissolved in filtered (0.2 μm) distilled water. The concentration of CpG was 1.2 μg/μL, which was monitored with NanoDrop 1000 Spectrophotometer by observing the absorbance at 260 nm (Extinction coefficient: 21130) (Thermo Scientific). Then 2 mg SWCNT was added into 2 mL of the prepared solution and ultrasonication was applied for 3 hours as described above. Then 4 mg of Lipid-PEG-NH₂ was added to the system and another 3 hours ultrasonication was applied. The dispersion was then dialyzed for 24 hours (15,000 Mw cut-off regenerated cellulose bag); water was changed every 2 hours in the first 8 hours. 200 μL of the final product was treated with DTT (50 mM, 200 μL) for 4 hours at room temperature to cleave the CpG, followed by ultracentrifugation at 15000 rpm for 1 hour to remove SWCNTs. The resultant free CpG in the supernatant was quantified by the UV absorbance at 260 nm (Extinction coefficient: 21130) to determine the final concentration of CpG in the SWCNTs/CpG conjugate (FIG. 31B). The final concentration of CpG was then adjusted to 1 μg/μL by adding filtered distilled water to the conjugates.

Zeta Potential Measurements

The zeta potential of the dilute suspensions (0.01 wt %) was measured with a Zeta potential analyzer (Zetapals, Brookhaven, N.Y., USA). Each sample was ultrasonicated for 1 h prior to analysis. The ionic strength was maintained at 10⁻³ M using NaCl.

Transmission Electron Microscopy

SWCNT dispersions were imaged using a FEI Tecnai 12 TEM equipped with a Gatan Ultrascan 2K CCD camera at an accelerating voltage of 120 kV.

### Example 14

### Characterizations of the Type II CNT-CpG Prepared According to Example 13

The synthesis and purification of Lipid-PEG-LC-SPDP-CpG was monitored by ¹H NMR in deuterated dimethylsulfoxide (DMSO) (FIG. 31A). A key diagnostic peak for C6-SS-CpG was (a) 3.27-3.33 ppm (O—CH₂—C) indicative of the deoxyribose backbone. Lipid-PEG-NH₂ was distinguished by (b) 3.47-3.52 (O—CH₂—C) from the PEG block and (c) 0.5-1.5 ppm (C—CH₂—C) from the lipid block. In the final product, Lipid-PEG-LC-SPDP-CpG, peaks (a), (b) and (c) ere present representing the conjugation of the DNA oligomer to the di-block copolymer. After conjugation, peak (a) was relatively unchanged while peak (b) shifted upfield and peak (c) was split into three separate peaks. Dynamic light scattering analysis revealed that Lipid-PEG-LC-SPDP-CpG formed micelles in DMSO. It was thus likely that in this micellar structure, the lipid chains were in several different environments, the PEG signal was shifted as a result of packing together in the micelle and the CpG was on the exterior so its signal was hardly changed. The synthesis was also monitored by ultraviolet-visible spectroscopy (UV-Vis). Lipid-PEG-NH₂ had no distinctive signal. When an aliquot of Lipid-PEG-LC-SPDP was analyzed, two peaks were observed at 230 nm and 275 nm, characteristic of 2-mercaptopyridine involved in a disulfide bond. Aliquots removed from the conjugation reaction between HS-CpG and Lipid-PEG-LC-SPDP prior to purification showed a peak at 345 nm, which is indicative of free 2-mercaptopyridine. When the intensity of this peak stopped increasing the reaction was judged to be complete. The final Lipid-PEG-LC-SPDP-CpG showed a strong peak at 260 nm, characteristic of DNA (FIG. 31B).

Delivery of therapeutics directly into the brain was constrained by the volume of liquid that can be injected because the brain has little free space and exerts a back pressure during injection. Thus, it would be beneficial to prepare SWCNTs/CpG as concentrated as possible. SWCNTs were known to be difficult to disperse at high concentration in aqueous solutions, with common surfactants such as sodium dodecyl sulfate (SDS) generally providing concentrations generally less than 0.01 mg/mL (20, 21).

Preparation of a SWCNT concentration of 1 mg/mL was attempted by sonicating the Lipid-PEG-LC-SPDP-CpG and SWCNTs together in an eppendorf tube for 3 hours. Surprisingly, nearly all of the SWCNTs settled to the bottom of the eppendorf tube after the sonication. Transmission electron microscopy (TEM) revealed that under these conditions only large aggregates of SWCNTs were formed (FIG. 31D).

To facilitate the dispersion of SWCNTs in aqueous solutions, SWCNTs were sonicated with various ratios of Lipid-PEG-NH₂: Lipid-PEG-LC-SPDP-CpG. A 1:1 ratio still produced large, unstable aggregates (FIG. 31E), but a 2:1 ratio provided a stable dispersion with no large aggregates observed by TEM (FIGS. 31F and 31G). Although both large and small bundles of SWCNTs were observed for the 2:1 ratio, it was possible that in solution the 2:1 ratio provided mostly the smaller bundles but during the drying process required for TEM the larger bundles were formed. Measurement with Image J showed that the average diameter of the small bundles of SWCNT-CpG conjugates was about 5 nm diameters (FIG. 31H). It has been reported that sufficient surface charge can allow for stable dispersal of SWCNT bundles in water (22). As the ζ potential of SWCNTs/CpG was −71.98±3.45 mV, it was possible, without being bound to any specific mechanism, that the electrostatic repulsion between the anionic CpG components of the Lipid-PEG-LC-SPDP-CpGs prevented their dense packing on the SWCNTs. The smaller, mildly cationic Lipid-PEG-NH₂ may be necessary to fill the “holes” between Lipid-PEG-LC-SPDP-CpG and provide the stable dispersion.

### Example 15

### In Vitro Efficacy Evaluation of the SWCNT-CpG-II Prepared According to Examples 13-14

The SWCNT-CpG-II prepared according to Examples 13-14 was tested for in vitro activity. RAW macrophage cells were treated with SWCNT-CpG-II, Lipid-PEG-LC-SPDP-CpG and RSS-CpG, respectively. The macrophage cells (RAW-Blue™) were stably transfected with a reporter construct that allowed for the measurement of NF-κB activation, as described supra. One day after SWCNT-CpG-II, free CpG, Lipid-PEG-LC-SPDP-CpG or PBS buffer was added to RAW-Blue cells, the NF-κB activity was measured using Quanti-Blue according to the manufacturer's instruction (InvivoGen).

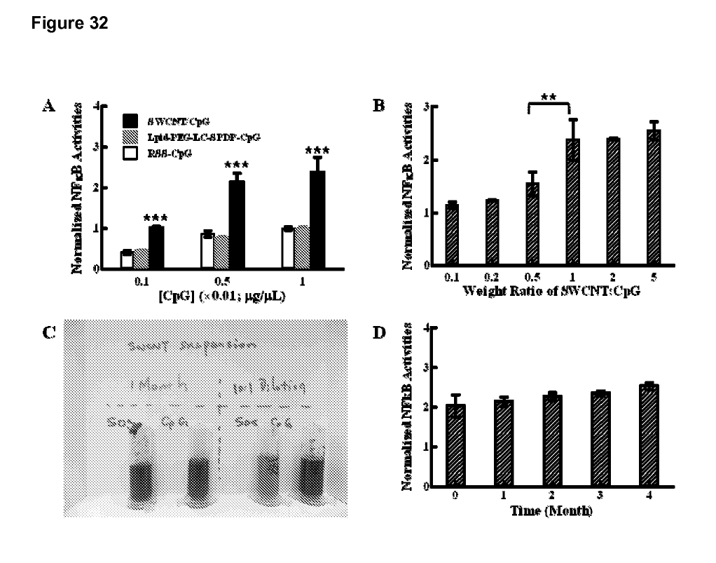

In order to avoid activity related to changes in the media, no more than 1% by volume SWCNT-CpG-II solution was added to the cells in media. The samples were ultrasonicated for 1 hour prior to each test. All data were normalized relative to the NF-κB activity of RSS-CpG at a concentration 0.01 μg/μL, ***: P<0.001, and **: P<0.01. As shown in FIG. 32A, Lipid-PEG-LC-SpDP-CpG had almost the same in vitro efficacy as RSS-CpG. SWCNT-CpG-II (SWCNT/CpG, FIG. 32A) had over 2× higher NF-κB activity than RSS-CpG at each concentration tested, suggesting that the SWCNT conjugation improved the efficacy of CpG.

This in vitro activity assay was then used to optimize the ratio of SWCNTs to the coating mixture (2:1 Lipid-PEG-NH₂/Lipid-PEG-LC-SPDP-CpG) (FIG. 32B). This was accomplished by adding various amounts of SWCNTs to the coating mixture (2:1 Lipid-PEG-NH₂/Lipid-PEG-LC-SPDP-CpG) with an effective concentration of CpG=1 mg/mL. Each mixture was ultrasonicated for 3 hours and then used without further purification. Thus, samples containing a low amount of SWCNTs consisted of a mixture of SWCNT-CpG-II and free CpG, while the samples with a large amount of SWCNTs consisted of SWCNT-CpG-II and additional poorly solubilized SWCNTs. Since free CpG had lower NF-κB activity than SWCNT/CpG, as the amount of free CpG in a sample decreased the NF-κB activity increased (FIG. 32B). The NF-κB activity increased in proportion to the amount of SWCNT up to the threshold weight ratio of SWCNT:CpG=1:1. This suggested that at the 1:1 weight ratio of SWCNT:CpG nearly all of the Lipid-PEG-LC-SPDP-CpG conjugated to SWCNT. When the weight ratio was increased beyond 1:1, no further increase in activity was observed. A preferred SWCNT-CpG-II used for in vivo testing and potential clinical translation was prepared as described supra using a mixture of SWCNT, Lipid-PEG-LC-SPDP-CpG, and Lipid-PEG-NH₂, wherein SWCNTs:CpG had about 1:1 weight ratio, and Lipid-PEG-LC-SPDP-CpG/Lipid-PEG-NH₂ had about 2:1 molar ratio.

### Example 16

### SWCNT-CpG-II Prepared According to Examples 13-15 Showed Good Stability

It has been reported that SDS-dispersed SWCNTs are not stable during prolonged storage (23). As a comparison, 1 mg of SWCNTs was dispersed in 1 mL of 2:1 Lipid-PEG-NH₂/Lipid-PEG-LC-SPDP-CpG ([CpG]=1 mg/mL) or SDS alone (3 mg/mL). After 1 month of storage at 4° C., there was no obvious change for SWCNT-CpG dispersion (CpG, FIG. 320), while precipitation was observed for the SWCNT-SDS dispersion (SDS, FIG. 32C). The supernatant from each sample was diluted 10:1 with water and showed the significantly higher concentration of SWCNTs still dispersed by Lipid-PEG-NH₂/Lipid-PEG-LC-SPDP-CpG (10:1 dilution, CpG, and SDS, FIG. 32C). The stability of the SWCNT-CpG-II was further evaluated by storing it at 4° C. for 4 months and testing its activity in vitro on the first day of each month. The activity remained the same for the duration of the testing (FIG. 32D). These results suggested that SWCNT-CpG-II prepared as described herein had sufficient stability to be considered for clinical translation.

### Example 17

### SWCNT-CpG-II Prepared According to Examples 13-15 Improved the Immunostimulatory Response of CpG

To further investigate how SWCNT conjugation improved the immunostimulatory response of CpG, human peripheral blood mononuclear cells (PBMCs) were incubated with SWCNT-CpG-II (1 mg/mL per well), free CpG (1 mg/mL per well) or PBS, and cytokine expression was evaluated by quantitative real-time polymerase chain reaction (qRT-PCR).

Human PBMCs Isolation

PBMCs from healthy adult donors at City of Hope were isolated by Ficoll density gradient centrifugation. Experiments with human materials were performed according to protocols approved by the institutional review committee. The purity of freshly isolated CD14+ monocytes was more than 80% as analyzed by flow cytometry. Monocytes were cultured in 12-well plates with RPMI 1640 medium containing 10% FBS. M-CSF and GM-CSF were purchased from PeproTech and used at a final concentration of 33 ng/ml. Cytokines were added to cultures every 3 days.

At various times, cells were collected and total RNA was isolated using the Trizol system (Invitrogen) followed by double DNase treatment and column purification using the Qiagen RNeasy Clean-up Protocol. Real-time PCR was performed in a TaqMan 5700 Sequence Detection System (Applied Biosystems) as described previously (25). PCR conditions were optimized such that a minimum of 10.000-fold range could be detected for each primer. GAPDH: 5′-TGCACCACCAACTGCTTAGC-3′ (SEQ ID NO: 17) and 3′-GGCATGGACTGTGGTCATGAG-5′ (SEQ ID NO: 18). TNFα: 5′-CCTGCCCCAATCCCTTTATT-3′ (SEQ ID NO: 19) and 3′-CCCTAAGCCCCCAATTCTCT-5′(SEQ ID NO: 20). IL-16: 5′-CCCTAAACAGATGAAGTGCTCCTT-3′ (SEQ ID NO: 21) and 3′-GTAGCTGGATGCCGCCAT-5′ (SEQ ID NO: 22). IL-12 (3: 5′-TGGAGTGCCAGGAGGACAGT-3′ (SEQ ID NO: 23) and 3′-TCTTGGGTGGGTCAGGTTTG-5′(SEQ ID NO: 24).

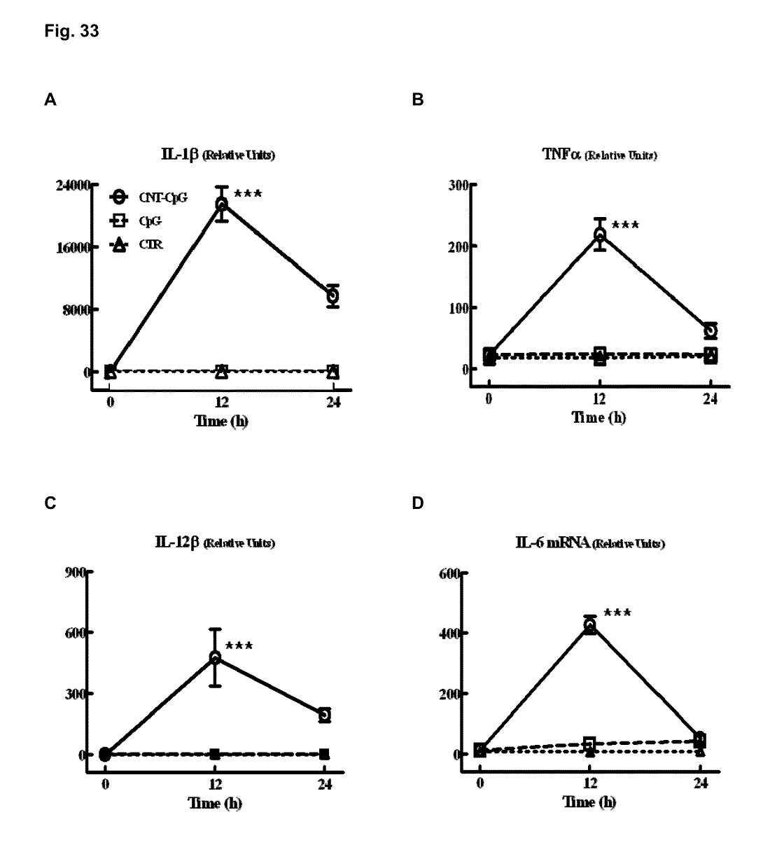

At the same low doses (1 mg/mL per well), a negligible response was observed for free CpG treatment while a significant upregulation of pro-inflammatory cytokines (including IL-1β, IL-12β, TNF-α and IL6 mRNA) was seen for SWCNT-CpG-II treatment, peaking at 12 hours after the administration (FIG. 33, *** P<0.001).

One possibility of the dramatic difference in the cytokine expression of the free CpG treated and SWCNT-CpG-II treated PBMCs may be that SWCNT-CpG-II delivered more CpG into the cells, while another possibility may be that SWCNT-CpG-II altered the manner in which the cells interacted with CpG.

Lipid-PEG-NH₂ was labeled with Alexa Fluor 555 (AF555) to produce Lipid-PEG-AF555. RSSCpG was prepared labeled with Cy5.5 such that the final tri-block polymer was Lipid-PEG-LC-SPDP-CpG^{5.5}. It was envisioned that the Lipid-Peg-AF555 would remain bound to the SWCNTs throughout the cell treatment, while the Cy5.5 would track the location of the CpG as the disulfide bond linking it to the construct was cleaved and the CpG was processed by the cells.

Confocal Microscopy Imaging

PBMCs were plated in 33 mm² dishes in 1 mL media and AF555-SWCNT/CpG^{5.5}, CpG^{5.5} or Lipid-PEG-AF555 were added. At various times intervals (4, 24 and 48 hours) the cells were washed three times with PBS and imaged by confocal microscopy. HeNe 678 nm and 543 nm lasers were used to excite Cy5.5 and AF555, respectively.

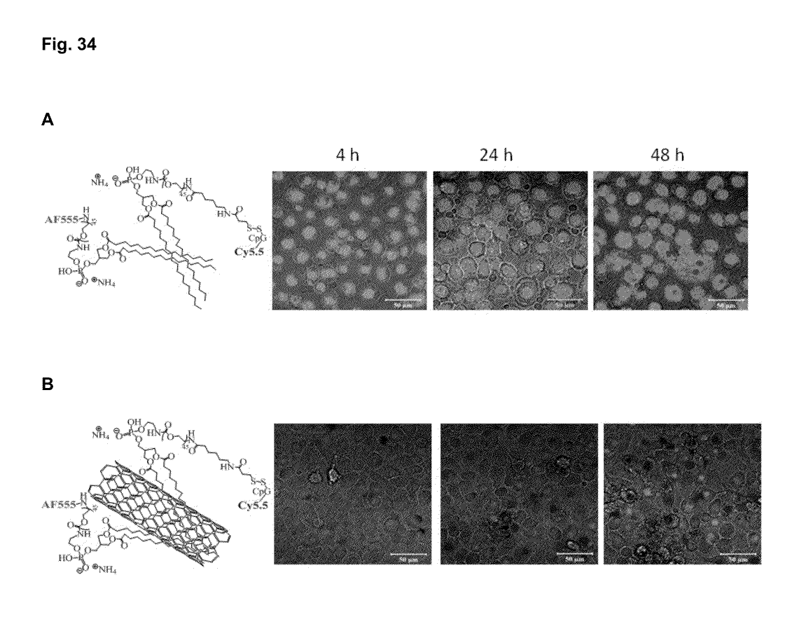

When human PBMCs were cultured in the presence of a mixture of free Lipid-PEG-AF555 (AF555 was false colored in red) and Lipid-PEG-CpG^{5.5} (Cy5.5 was false colored in blue) both polymers were quickly taken up by human PBMCs and remained in the cytoplasm without being consumed for at least 48 hours (FIG. 34A). On the other hand, human PBMCs took up the AF555-SWCNT-CpG^{5.5}-II at a significantly slower rate, and as can be seen in the images by a color change from purple to red, the signal from the AF555 persisted for at least 48 hours, but the signal from the Cy5.5 faded gradually (FIG. 34B). This observation suggested that the SWCNT-CNT-II functioned primarily by altering the processing of CpG by immune cells.

### Example 18

### In Vivo Efficacy Evaluation of the SWCNT-CpG-II Prepared According to Examples 13-15

Tumor Implantation, Treatment and Tumor Rechallenge

All animals were housed and handled according to the guidelines of City of Hope Institutional Animal Care and Use Committee. Intracranial tumor implantation was done as described previously (24). GL261.luc cells were harvested by trypsinization, counted, and resuspended in PBS. Female C57BL/6 mice weight 15 to 25 g were anesthetized by intraperotenial administration of ketamine (132 mg/kg) and xylazine (8.8 mg/kg) and immobilized in a stereostactic head frame. Through a small burr hold, 3 μL of PBS containing 1×10⁵ tumor cells were injected unilaterally at the coronal suture, 1 mm laceral to the midline, and 3 mm deep into the frontal lobes, using a Hamilton syringe (Fisher Scientific). Four days after intracranial implantation of tumor cells, PBS (control, 5 μL), CpG (5 μg/5 μL PBS) and SWCNTs/CpG (5 μg SWCNT/5 μg CpG/5 μL PBS) were administered through the initial burr hole aiming to target the tumor site (5 mice per group). For tumor rechallenge experiments, naive or GL261-bearing mice that had survived for at least 3 months after the initial SWCNT/CpG treatment were rechallenged with a subcutaneous injection of GL261 (1×10⁶ cells). Tumor growth was monitored every other day for 15 days using calipers.

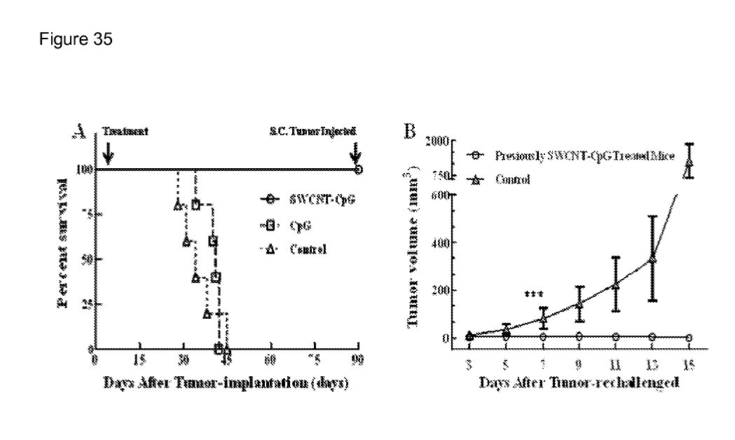

SWCNT-CpG-II prepared following the optimized protocol described in Examples 13-15 was evaluated for the treatment of a murine model of glioma. Mice bearing 4-day-old intracranial GL261.luc gliomas were given a single intratumoral injection of PBS (control), free RSS-CpG (5 mg CpG/10 mL PBS) and SWCNT-CpG-II (5 mg SWCNT/5 mg CpG/10 mL PBS). The mice were then monitored for up to 90 days, and mice that became ill were humanely euthanized. The brains of all euthanized mice were evaluated to determine if the glioma was the likely cause of illness. As shown in FIG. 35A, a single low-dose injection of free CpG did not significantly extend the survival of the mice. All of the mice in this group had large gliomas at the time of euthanization. In contrast, all of the mice treated with a single injection of SWCNTs/CpG survived at the 90 day conclusion of the experiment.

Thus, the mice that had survived 90 days after treatment with SWCNT-CpG were injected with GL261 cells subcutaneously in their flank, as were a new set of control mice. Caliper measurements showed that while the subcutaneous tumors grew quickly in the control mice, the mice previously treated with SWCNT-CpG-II had no detectable tumors remaining after 15 days (FIG. 35B). At this point these mice were euthanized, and no tumors were detected either in the brain or in the flank. These results demonstrated that just a single injection of the optimized formulation of SWCNT-CpG-II improved the survival of and induced a systemic immune response in the treated mice.

The following references are incorporated herein by reference:
1. Galea I, Bechmann I, Perry V H. What is immune privilege (not)? Trends Immunol. 2007; 28:12-8.
2. Prins R M, Shu C J, Radu C G, Vo D D, Khan-Farooqi H, Soto H, et al. Anti-tumor activity and trafficking of self, tumor-specific T cells against tumors located in the brain. Cancer Immunol Immunother. 2008; 57:1279-89.
3. Parney I F, Hao C, Petruk K C. Glioma immunology and immunotherapy. Neurosurgery. 2000; 46:778-91; discussion 91-2.
4. Yang L, Ng K Y, Lillehei K O. Cell-mediated immunotherapy: a new approach to the treatment of malignant glioma. Cancer Control. 2003; 10:138-47.
5. Sampson J H, Heimberger A B, Archer G E, Aldape K D, Friedman A H, Friedman H S, et al. Immunologic Escape After Prolonged Progression-Free Survival With Epidermal Growth Factor Receptor Variant III Peptide Vaccination in Patients With Newly Diagnosed Glioblastoma. Journal of Clinical Oncology. 2010; 28:4722-9.
6. Carpentier A, Laigle-Donadey F, Zohar S, Capelle L, Behin A, Tibi A, et al. Phase 1 trial of a CpG oligodeoxynucleotide for patients with recurrent glioblastoma. Neuro Oncol. 2006; 8:60-6.
7. Dalpke A H, Schafer M K, Frey M, Zimmermann S, Tebbe J, Weihe E, et al. Immunostimulatory CpG-DNA activates murine microglia. J. Immunol. 2002; 168:4854-63.
8. El Andaloussi A, Sonabend A M, Han Y, Lesniak M S. Stimulation of TLR9 with CpG ODN enhances apoptosis of glioma and prolongs the survival of mice with experimental brain tumors. Glia. 2006; 54:526-35.
9. Jahrsdorfer B, Weiner G J. CpG oligodeoxynucleotides as immunotherapy in cancer. Update Cancer Ther. 2008; 3:27-32.
10. Weber J S, Zarour H, Redman B, Trefzer U, O'Day S, van den Eertwegh A J, et al. Randomized phase 2/3 trial of CpG oligodeoxynucleotide PF-3512676 alone or with dacarbazine for patients with unresectable stage III and IV melanoma. Cancer. 2009; 115:3944-54.
11. Carpentier A, Metellus P, Ursu R, Zohar S, Lafitte F, Barrie M, et al. Intracerebral administration of CpG oligonucleotide for patients with recurrent glioblastoma: a phase II study. Neuro Oncol. 2010; 12:401-8.
12. Rosi N L, Giljohann D A, Thaxton C S, Lytton-Jean A K, Han M S, Mirkin C A. Oligonucleotide-modified gold nanoparticles for intracellular gene regulation. Science. 2006; 312:1027-30.
13. Daftarian P, Song G Y, Ali S, Faynsod M, Longmate J, Diamond D J, et al. Two distinct pathways of immuno-modulation improve potency of p53 immunization in rejecting established tumors. Cancer Res. 2004; 64:5407-14.
14. Alizadeh D, Zhang L, Brown C E, Farrukh O, Jensen M C, Badie B. Induction of anti-glioma NK cell response following multiple low-dose intracerebral CpG therapy. Clin Cancer Res. 2010; 16:3399-408.
15. Kateb B, Yamamoto V, Alizadeh D, Zhang L, Manohara H M, Bronikowski M J, et al. Multi-walled carbon nanotube (MWCNT) synthesis, preparation, labeling, and functionalization. Methods Mol Biol. 2010; 651:307-17.
16. Kateb B, Van Handel M, Zhang L, Bronikowski M J, Manohara H, Badie B. Internalization of MWCNTs by microglia: possible application in immunotherapy of brain tumors. Neuroimage. 2007; 37 Suppl 1:S9-17.
17. Manohara H M, Wong E W, Schlecht E, Hunt B D, Siegel P H. Carbon nanotube Schottky diodes using Ti-Schottky and Pt-Ohmic contacts for high frequency applications. Nano Lett. 2005; 5:1469-74.
18. Liu Z, Tabakman S M, Chen Z, Dai H. Preparation of carbon nanotube bioconjugates for biomedical applications. Nat Protoc. 2009; 4:1372-82.
19. Kam N R, Liu Z, Dai H. Functionalization of carbon nanotubes via cleavable disulfide bonds for efficient intracellular delivery of siRNA and potent gene silencing. J Am Chem Soc. 2005; 127:12492-3.
20. Zhang L, Alizadeh D, Van Handel M, Kortylewski M, Yu H, Badie B. Stat3 inhibition activates tumor macrophages and abrogates glioma growth in mice. Glia. 2009; 57:1458-67.
21. Badie B, Schartner J M. Flow cytometric characterization of tumor-associated macrophages in experimental gliomas. Neurosurgery. 2000; 46:957-61; discussion 61-2.
22. Larocque D, Sanderson N S, Bergeron J, Curtin J F, Girton J, Wibowo M, et al. Exogenous fms-like tyrosine kinase 3 ligand overrides brain immune privilege and facilitates recognition of a neo-antigen without causing autoimmune neuropathology. Proc Natl Acad Sci USA. 2010; 107:14443-8.
23. Pellegatta S, Poliani P L, Stucchi E, Corno D, Colombo C A, Orzan F, et al. Intra-tumoral dendritic cells increase efficacy of peripheral vaccination by modulation of glioma microenvironment. Neuro Oncol. 2010; 12:377-88.
24. Yamanaka R, Homma J, Yajima N, Tsuchiya N, Sano M, Kobayashi T, et al. Clinical Evaluation of Dendritic Cell Vaccination for Patients with Recurrent Glioma: Results of a Clinical Phase I/II Trial. Clinical Cancer Research. 2005; 11:4160-7.
25. Hu J, Yuan X, Belladonna M L, Ong J M, Wachsmann-Hogiu S, Farkas D L, et al. Induction of Potent Antitumor Immunity by Intratumoral Injection of Interleukin 23—Transduced Dendritic Cells. Cancer Research. 2006; 66:8887-96.
26. Gomez G G, Kruse C A. Mechanisms of malignant glioma immune resistance and sources of immunosuppression. Gene therapy & molecular biology 2006; 10(A):133-46.
27. Vollmer J, Krieg A M. Immunotherapeutic applications of CpG oligodeoxynucleotide TLR9 agonists. Adv Drug Deliv Rev 2009; 61(3):195-204.
28. Olson J K, Miller S D. Microglia initiate central nervous system innate and adaptive immune responses through multiple TLRs. J Immunol 2004; 173(6):3916-24.
29. Carpentier A F, Auf G, Delattre J Y. CpG-oligonucleotides for cancer immunotherapy: review of the literature and potential applications in malignant glioma. Front Biosci 2003; 8:e115-27.
30. Bianco A, Hoebeke J, Kostarelos K, Prato M, Partidos C D. Carbon nanotubes: on the road to deliver. Curr Drug Deliv 2005; 2(3):253-9.
31. Pulskamp K, Diabate S, Krug H F. Carbon nanotubes show no sign of acute toxicity but induce intracellular reactive oxygen species in dependence on contaminants. Toxicol Lett 2007; 168(1):58-74.
32. Schipper M L, Nakayama-Ratchford N, Davis C R, et al. A pilot toxicology study of single-walled carbon nanotubes in a small sample of mice. Nat Nano 2008; 3(4):216-21.
33. VanHandel M, Alizadeh D, Zhang L, et al. Selective uptake of multi-walled carbon nanotubes by tumor macrophages in a murine glioma model. J Neuroimmunol 2009; 208(1-2):3-9.
34. McDevitt M R, Chattopadhyay D, Kappel B J, et al. Tumor targeting with antibody-functionalized, radiolabeled carbon nanotubes. J Nucl Med 2007; 48(7):1180-9.
35. Singh R, Pantarotto D, Lacerda L, et al. Tissue biodistribution and blood clearance rates of intravenously administered carbon nanotube radiotracers. Proceedings of the National Academy of Sciences of the United States of America 2006; 103(9):3357-62.
36. Wang H, Wang J, Deng X, et al. Biodistribution of carbon single-wall carbon nanotubes in mice. J Nanosci Nanotechnol 2004; 4(8):1019-24.
37. Liu Z, Cai W, He L, et al. In vivo biodistribution and highly efficient tumour targeting of carbon nanotubes in mice. Nat Nanotechnol 2007; 2(1):47-52.
38. Villa C H, McDevitt M R, Escorcia F E, et al. Synthesis and Biodistribution of Oligonucleotide-Functionalized, Tumor-Targetable Carbon Nanotubes. Nano letters 2008.
39. Alizadeh D, Zhang L, Hwang J, Schluep T, Badie B. Tumor-associated macrophages are predominant carriers of cyclodextrin-based nanoparticles into gliomas. Nanomedicine 2010; 6(2):382-90.
40.Carpentier A F, Chen L, Maltonti F, Delattre J Y. Oligodeoxynucleotides containing CpG motifs can induce rejection of a neuroblastoma in mice. Cancer Res 1999; 59(21):5429-32.
41. Carpentier A F, Xie J, Mokhtari K, Delattre J Y. Successful treatment of intracranial gliomas in rat by oligodeoxynucleotides containing CpG motifs. Clin Cancer Res 2000; 6(6):2469-73.
42. Wu A, Oh S, Gharagozlou S, et al. In vivo vaccination with tumor cell lysate plus CpG oligodeoxynucleotides eradicates murine glioblastoma. J Immunother 2007; 30(8):789-97.
43. Grauer O M, Molling J W, Bennink E, et al. TLR ligands in the local treatment of established intracerebral murine gliomas. J Immunol 2008; 181(10):6720-9.
44. Wilson K D, de Jong S D, Tam Y K. Lipid-based delivery of CpG oligonucleotides enhances immunotherapeutic efficacy. Adv Drug Deliv Rev 2009; 61(3):233-42.
45. Bourquin C, Anz D, Zwiorek K, et al. Targeting CpG oligonucleotides to the lymph node by nanoparticles elicits efficient antitumoral immunity. J Immunol 2008; 181(5):2990-8.
46. Chikh G, de Jong S D, Sekirov L, et al. Synthetic methylated CpG ODNs are potent in vivo adjuvants when delivered in liposomal nanoparticles. Int Immunol 2009; 21(7):757-67.
47. de Jong S D, Basha G, Wilson K D, et al. The immunostimulatory activity of unmethylated and methylated CpG oligodeoxynucleotide is dependent on their ability to colocalize with TLR9 in late endosomes. J Immunol; 184(11):6092-102.
48. Malyala P, O'Hagan D T, Singh M. Enhancing the therapeutic efficacy of CpG oligonucleotides using biodegradable microparticles. Adv Drug Deliv Rev 2009; 61(3):218-25.
49. Schreiber H A, Prechl J, Jiang H, et al. Using carbon magnetic nanoparticles to target, track, and manipulate dendritic cells. J Immunol Methods; 356(1-2):47-59.
50. Krishnamachari Y, Salem A K. Innovative strategies for co-delivering antigens and CpG oligonucleotides. Adv Drug Deliv Rev 2009; 61(3):205-17.
51. Partidos C D, Hoebeke J, Wieckowski S, et al. Immunomodulatory consequences of ODN CpG-polycation complexes. Methods 2009; 49(4):328-33.
52. Wagner H. The sweetness of the DNA backbone drives Toll-like receptor 9. Curr Opin Immunol 2008; 20(4):396-400.
53. Haas T, Metzger J, Schmitz F, et al. The DNA sugar backbone 2′ deoxyribose determines toll-like receptor 9 activation. Immunity 2008; 28(3):315-23.
54. Yasuda K, Rutz M, Schlatter B, et al. CpG motif-independent activation of TLR9 upon endosomal translocation of “natural” phosphodiester DNA. Eur J Immunol 2006; 36(2):431-6.
55. Raffa V, Ciofani G, Vittorio O, Riggio C, Cuschieri A. Physicochemical properties affecting cellular uptake of carbon nanotubes. Nanomedicine (Lond) 2010; 5(1):89-97.
56. Allen B L, Kichambare P D, Gou P, et al. Biodegradation of Single-Walled Carbon Nanotubes through Enzymatic Catalysis. Nano Letters 2008; 8(11):3899-903.
57.Chou C-C, Hsiao H-Y, Hong Q-S, et al. Single-Walled Carbon Nanotubes Can Induce Pulmonary Injury in Mouse Model. Nano Letters 2008; 8(2):437-45.
58. De La Zerda A, Zavaleta C, Keren S, et al. Carbon nanotubes as photoacoustic molecular imaging agents in living mice. Nat Nano 2008; 3(9):557-62.
59. Liu Z, Davis C, Cai W, He L, Chen X, Dai H. Circulation and long-term fate of functionalized, biocompatible single-walled carbon nanotubes in mice probed by Raman spectroscopy. Proc Natl Acad Sci USA 2008; 105(5):1410-5.
60.Al-Jamal K T, Toma F M, Yilmazer A, et al. Enhanced cellular internalization and gene silencing with a series of cationic dendron-multiwalled carbon nanotube:siRNA complexes. FASEB J 2010.
61. Stupp, R.; Mason, W. P.; van den Bent, M. J.; Weller, M.; Fisher, B.; Taphoorn, M. J.; Belanger, K.; Brandes, A. A.; Marosi, C.; Bogdahn, U.; Curschmann, J.; Janzer, R. C.; Ludwin, S. K.; Gorlia, T.; Allgeier, A.; Lacombe, D.; Cairncross, J. G.; Eisenhauer, E.; Mirimanoff, R. O., Radiotherapy plus concomitant and adjuvant temozolomide for glioblastoma. N Engl J Med 2005, 352 (10), 987-996.
62. Meng, Y.; Kujas, M.; Marie, Y.; Paris, S.; Thillet, J.; Delattre, J. Y.; Carpentier, A. F., Expression of TLR9 within human glioblastoma. J Neurooncol 2008, 88 (1), 19-25.
63. Hussain, S. F.; Yang, D.; Suki, D.; Grimm, E.; Heimberger, A. B., Innate immune functions of microglia isolated from human glioma patients. J Transl Med 2006, 4, 15.
64. Hussain, S. F.; Yang, D.; Suki, D.; Aldape, K.; Grimm, E.; Heimberger, A. B., The role of human glioma-infiltrating microglia/macrophages in mediating antitumor immune responses. Neuro Oncol 2006, 8 (3), 261-279.
65. Bsibsi, M.; Ravid, R.; Gveric, D.; van Noort, J. M., Broad expression of Toll-like receptors in the human central nervous system. J Neuropathol Exp Neurol 2002, 61 (11), 1013-1021.
66. Klinman, D. M., Adjuvant activity of CpG oligodeoxynucleotides. Int Rev Immunol 2006, 25 (3-4), 135-154.
67. Goldstein, M. J.; Varghese, B.; Brody, J. D.; Rajapaksa, R.; Kohrt, H.; Czerwinski, D. K.; Levy, S.; Levy, R., A CpG-loaded tumor cell vaccine induces antitumor CD4+ T cells that are effective in adoptive therapy for large and established tumors. Blood 2011, 117 (1), 118-127.
68. Baines, J.; Celis, E., Immune-mediated tumor regression induced by CpG-containing oligodeoxynucleotides. Clinical Cancer Research 2003, 9 (7), 2693-2700.
69. Wagner, H., The immunobiology of the TLR9 subfamily. Trends in immunology 2004, 25 (7), 381-386.
70.Kolosnjaj-Tabi, J.; Hartman, K. B.; Boudjemaa, S.; Ananta, J. S.; Morgant, G.; Szwarc, H.; Wilson, L. J.; Moussa, F., In vivo behavior of large doses of ultrashort and full-length single-walled carbon nanotubes after oral and intraperitoneal administration to Swiss mice. ACS Nano 2010, 4 (3), 1481-1492.
71. Kam, N. W.; Liu, Z.; Dai, H., Carbon nanotubes as intracellular transporters for proteins and DNA: an investigation of the uptake mechanism and pathway. Angew Chem Int Ed Engl 2006, 45 (4), 577-581.
72. Zhao, D.; Alizadeh, D.; Zhang, L.; Liu, W.; Farrukh, 0.; Manuel, E.; Diamond, D. J.; Badie, B., Carbon nanotubes enhance CpG uptake and potentiate antiglioma immunity. Clinical cancer research: an official journal of the American Association for Cancer Research 2011, 17 (4), 771-782.
73. Nam, C. W.; Kang, S. J.; Kang, Y. K.; Kwak, M. K., Cell growth inhibition and apoptosis by SDS-solubilized single-walled carbon nanotubes in normal rat kidney epithelial cells. Arch Pharm Res 2011, 34 (4), 661-669.
74. O'Connell, M. J.; Bachilo, S. M.; Huffman, C. B.; Moore, V. C.; Strano, M. S.; Haroz, E. H.; Rialon, K. L.; Boul, P. J.; Noon, W. H.; Kittrell, C.; Ma, J. P.; Hauge, R. H.; Weisman, R. B.; Smalley, R. E., Band gap fluorescence from individual single-walled carbon nanotubes. Science 2002, 297 (5581), 593-596.
75. Hu, H.; Yu, A. P.; Kim, E.; Zhao, B.; Itkis, M. E.; Bekyarova, E.; Haddon, R. C., Influence of the zeta potential on the dispersability and purification of single-walled carbon nanotubes. J Phys Chem B 2005, 109 (23), 11520-11524.
76. Li, L. J.; Nicholas, R. J.; Chen, C. Y.; Darton, R. C.; Baker, S. C., Comparative study of photoluminescence of single-walled carbon nanotubes wrapped with sodium dodecyl sulfate, surfactin and polyvinylpyrrolidone. Nanotechnology 2005, 16 (5), S202-S205.
77. Manuel E R, Blache C A, Paquette R, Kaltcheva T I, Ishizaki H, Ellenhorn J D, et al. Enhancement of cancer vaccine therapy by systemic delivery of a tumor-targeting Salmonella-based STAT3 shRNA suppresses the growth of established melanoma tumors. Cancer Res 2011; 71:4183-91.

What is claimed is:

1. A method comprising administering a therapeutically effective amount of nanoparticle (NANO) conjugated CpG (NANO-CpG) or a pharmaceutical composition thereof to a subject, wherein the NANO-CpG administration improves the CpG delivery into brain tumor associated inflammatory cells in the subject compared to an administration of free CpG of the same amount.

2. The method of claim 1, wherein the brain tumor is gliomas or metastatic brain tumor.

3. The method of claim 1, wherein the inflammatory cells are selected from the group consisting of microglia and macrophages (MG/MP), NK cells, and dendritic cells (DC).

4. The method of claim 1, wherein the NANO is selected from the group consisting of carbon nanotube (CNT), gold, iron, silica, organic polymers, and carbon nanomaterials.

5. The method of claim 4, wherein the CNT is selected from the group consisting of single-walled CNTs (SWCNTs), double-walled CNTs (DWCNTs), triple-walled CNTs (TWCNTs), multi-walled CNTS (MWCNTs), and any combinations thereof.

6. The method of claim 4, wherein the carbon nanomaterials are selected from the group consisting of fullerenes, rapheme, nanohorns, nanodiamond, and any combinations thereof.

7. The method of claim 1, wherein the conjugation between CNT and CpG involves covalent interactions, non-covalent interactions or a combination thereof.

8. The method of claim 1, wherein the NANO-CpG is a type II NANO-CpG prepared by a method comprising modifying a NANO with a NANO modifying agent (NANOMA) conjugated CpG (NANOMA-CpG).

9. The method of claim 8, wherein the NANO comprises SWCNT.

10. The method of claim 9, wherein:
the NANOMA is Lipid-PEG-NH₂: 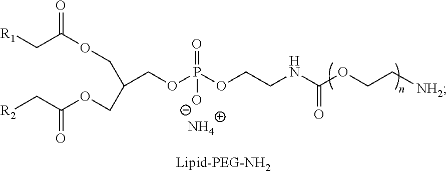
R₁ is Cₐ alkyl;
R₂ is C_{b} alkyl group;
a and b are the same or different and are independently selected from the group consisting of 10, 11, 12, 13, 14, 15, 16, 17, and 18; and
n=30, 31 . . . 44, 45, 46 . . . or 100.

11. The method of claim 10, wherein the type II NANO-CpG is a SWCNT-CpG-II prepared by a method comprising the following steps:
i) reacting Lipid-PEG-NH₂ with a thiolated CpG (sCpG) and an optional linker L to provide Lipid-PEG-L-CpG; and
ii) sonicating an aqueous mixture of SWCNTs, Lipid-PEG-L-CpG and Lipid-PEG-NH₂ to provide the SWCNT-CpG-II.

12. The method of claim 11, wherein the weight ratio of SWCNTs and Lipid-PEG-L-CpG is about 1:1.

13. The method of claim 11, wherein the molar ratio of Lipid-PEG-NH₂:Lipid-PEG-L-CpG is about 2:1.

14. A method for treating and/or preventing a brain tumor in a subject comprising administering a therapeutically effective amount of NANO-CpG or a pharmaceutical composition thereof to the subject.

15. The method of claim 14, wherein the brain tumor is gliomas or metastatic brain tumor.

16. The method of claim 14, wherein the inflammatory cells are selected from the group consisting of microglia and macrophages (MG/MP), NK cells, and dendritic cells (DC).

17. The method of claim 14, wherein the NANO is selected from the group consisting of carbon nanotube (CNT), gold, iron, silica, organic polymers, and carbon nanomaterials.

18. The method of claim 17, wherein the CNT is selected from the group consisting of single-walled CNTs (SWCNTs), double-walled CNTs (DWCNTs), triple-walled CNTs (TWCNTs), multi-walled CNTS (MWCNTs), and any combinations thereof.

19. The method of claim 17, wherein the carbon nanomaterials are selected from the group consisting of fullerenes, rapheme, nanohorns, nanodiamond, and any combinations thereof.

20. The method of claim 14, wherein the conjugation between CNT and CpG involves covalent interactions, non-covalent interactions or a combination thereof.

21. The method of claim 14, wherein the NANO-CpG is a type II NANO-CpG prepared by a method comprising modifying a NANO with a NANO modifying agent (NANOMA) conjugated CpG (NANOMA-CpG).

22. The method of claim 21, wherein the NANO comprises SWCNT.

23. The method of claim 22, wherein:
the NANOMA is Lipid-PEG-NH₂: 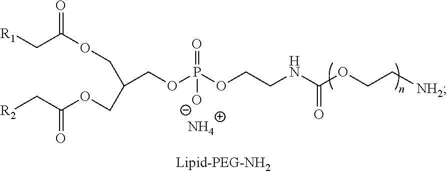
R₁ is Cₐ alkyl;
R₂ is C_{b} alkyl group;
a and b are the same or different and are independently selected from the group consisting of 10, 11, 12, 13, 14, 15, 16, 17, and 18; and
n=30, 31 . . . 44, 45, 46 . . . or 100.

24. The method of claim 23, wherein the type II NANO-CpG is a SWCNT-CpG-II prepared by a method comprising the following steps:
i) reacting Lipid-PEG-NH₂ with a thiolated CpG (sCpG) and an optional linker L to provide Lipid-PEG-L-CpG; and
ii) sonicating an aqueous mixture of SWCNTs, Lipid-PEG-L-CpG and Lipid-PEG-NH₂ to provide the SWCNT-CpG-II.

25. The method of claim 24, wherein the weight ratio of SWCNTs and Lipid-PEG-L-CpG is about 1:1.

26. The method of claim 24, wherein the molar ratio of Lipid-PEG-NH₂:Lipid-PEG-L-CpG is about 2:1.

27. A composition prepared by a method comprising:
i) reacting Lipid-PEG-NH₂ with a thiolated CpG (sCpG) and an optional linker L to provide Lipid-PEG-L-CpG; and
ii) sonicating an aqueous mixture of SWCNTs, Lipid-PEG-L-CpG and Lipid-PEG-NH₂ to provide an aqueous type II SWCNT-CpG dispersion.

28. The composition of claim 27, wherein the weight ratio of SWCNTs and Lipid-PEG-L-CpG is about 1:1.

29. The composition of claim 27, wherein the molar ratio of Lipid-PEG-NH₂:Lipid-PEG-L-CpG is at least about 2:1.

30. The composition of claim 27, wherein the aqueous type II SWCNT-CpG dispersion is stable for at least 4 months when stored at 4° C.

31. A composition comprising:
a) a nanoparticle conjugated CpG (NANO-CpG) or a pharmaceutical composition thereof;
b) a nanoparticle modifying agent (NANOMA) conjugated CpG (NANOMA-CpG) or a pharmaceutical composition thereof; or
c) a combination of a) and b); wherein the conjugation between the NANOMA and CpG is optionally through a linker L.

32. The composition of claim 31, wherein the NANO is a CNT.

33. The composition of claim 31, wherein the CNT is a SWCNT.

34. The composition of claim 31, further comprising the NANOMA, and wherein the molar ratio of NANOMA:NANOMA-CpG is at least about 2:1.

35. The composition of claim 31, wherein the NANO-CpG is SWCNT-Lipid-PEG-LC-SPDP-CpG and the NANOMA is Lipid-PEG-NH₂.

36. The composition of claim 32, where the composition treats or prevents brain tumor.


SEQUENCE LISTING (24 sequences)

SEQ ID NO: 1 - DNA, 21 bp, Artificial Sequence
  CpG sequence
tgactgtaacgttcgagatga

SEQ ID NO: 2 - DNA, 26 bp, Artificial Sequence
  CpG sequence
taaacgttataacgttatgacgtcat

SEQ ID NO: 3 - DNA, 21 bp, Artificial Sequence
  thiolated CpG
tgactgtaacgttcgagatga

SEQ ID NO: 4 - DNA, 26 bp, Artificial Sequence
  thiolated CpG
taaacgttataacgttatgacgtcat

SEQ ID NO: 5 - DNA, 21 bp, Artificial Sequence
  thiolated CpG
tgactgtaacgttcgagatga

SEQ ID NO: 6 - DNA, 21 bp, Artificial Sequence
  thiolated CpG
tgactgtaaggttagagatga

SEQ ID NO: 7 - DNA, 20 bp, Artificial Sequence
  RT-PCR primer
gttagtggggtctcgctctg

SEQ ID NO: 8 - DNA, 18 bp, Artificial Sequence
  RT-PCR primer
ggcaaattcaacggcaca

SEQ ID NO: 9 - DNA, 24 bp, Artificial Sequence
  RT-PCR primer
cagaccctcacactcagatcatct

SEQ ID NO: 10 - DNA, 21 bp, Artificial Sequence
  RT-PCR primer
cctccacttggtggtttgcta

SEQ ID NO: 11 - DNA, 20 bp, Artificial Sequence
  RT-PCR primer
agaggtggactggactcccg

SEQ ID NO: 12 - DNA, 20 bp, Artificial Sequence
  RT-PCR primer
agtctcgcctcctttgtggc

SEQ ID NO: 13 - DNA, 19 bp, Artificial Sequence
  RT-PCT primer
agggctgtctggagtcctc

SEQ ID NO: 14 - DNA, 19 bp, Artificial Sequence
  RT-PCR primer
gaccagccgccgccgcagg

SEQ ID NO: 15 - DNA, 26 bp, Artificial Sequence
  dithiolated CpG oligonucleotides
taaacgttataacgttatgacgtcat

SEQ ID NO: 16 - DNA, 26 bp, Artificial Sequence
  Cy5.5 labeled CpG oligonucleotides
taaacgttataacgttatgacgtcat

SEQ ID NO: 17 - DNA, 20 bp, Artificial Sequence
  RT-PCR primer
tgcaccaccaactgcttagc

SEQ ID NO: 18 - DNA, 21 bp, Artificial Sequence
  RT-PCR primer
ggcatggactgtggtcatgag

SEQ ID NO: 19 - DNA, 20 bp, Artificial Sequence
  RT-PCR primer
cctgccccaatccctttatt

SEQ ID NO: 20 - DNA, 20 bp, Artificial Sequence
  RT-PCR primer
ccctaagcccccaattctct

SEQ ID NO: 21 - DNA, 24 bp, Artificial Sequence
  RT-PCR primer
ccctaaacagatgaagtgctcctt

SEQ ID NO: 22 - DNA, 18 bp, Artificial Sequence
  RT-PCR primer
gtagctggatgccgccat

SEQ ID NO: 23 - DNA, 20 bp, Artificial Sequence
  RT-PCR primer
tggagtgccaggaggacagt

SEQ ID NO: 24 - DNA, 20 bp, Artificial Sequence
  RT-PCR primer
tcttgggtgggtcaggtttg